SEQUENCE LISTING (38 sequences)

SEQ ID NO: 1 - protein, 7 aa, Artificial Sequence
  non-naturally occurring molecule
  MISC_FEATURE (6)..(6): X can be H or R
  MISC_FEATURE (7)..(7): X can be N or Q
LTAFLXX

SEQ ID NO: 2 - protein, 7 aa, Artificial Sequence
  non-naturally occurring molecule
TAQILNW

SEQ ID NO: 3 - protein, 7 aa, Artificial Sequence
  non-naturally occurring molecule
AQILNWI

SEQ ID NO: 4 - protein, 7 aa, Artificial Sequence
  non-naturally occurring molecule
LAAALML

SEQ ID NO: 5 - protein, 21 aa, Artificial Sequence
  non-natural molecule
RLTAFLHNRRPRLTAFLHNRR

SEQ ID NO: 6 - protein, 21 aa, Artificial Sequence
  non-natural molecule
RLTAFLRQRRPRLTAFLRQRR

SEQ ID NO: 7 - protein, 21 aa, Artificial Sequence
  non-natural molecule
RLTAFLHNRRPRLTAFLRQRR

SEQ ID NO: 8 - protein, 21 aa, Artificial Sequence
  non-natural molecule
RLTAFLRQRRPRLTAFLHNRR

SEQ ID NO: 9 - protein, 7 aa, Artificial Sequence
  non-naturally occurring molecule
LTAFLHN

SEQ ID NO: 10 - protein, 7 aa, Artificial Sequence
  non-naturally occurring molecule
LTAFLRQ

SEQ ID NO: 11 - protein, 5 aa, Artificial Sequence
  non-natural molecule
LTAFL

SEQ ID NO: 12 - protein, 21 aa, Artificial Sequence
  NDM1-1
RTAQILNWRRPRTAQILNWRR

SEQ ID NO: 13 - protein, 21 aa, Artificial Sequence
  NDM1-2
RLAAALMLRRPRAQILNWIRR

SEQ ID NO: 14 - protein, 7 aa, Artificial Sequence
  non-natural molecule
TAQILNW

SEQ ID NO: 15 - protein, 6 aa, Artificial Sequence
  APR
FFAAFC

SEQ ID NO: 16 - protein, 7 aa, Artificial Sequence
  APR
FFAAFCL

SEQ ID NO: 17 - protein, 6 aa, Artificial Sequence
  APR
LLLTTI

SEQ ID NO: 18 - protein, 7 aa, Artificial Sequence
  APR
LLLTTIG

SEQ ID NO: 19 - protein, 5 aa, Artificial Sequence
  APR
LLTLA

SEQ ID NO: 20 - protein, 6 aa, Artificial Sequence
  APR
LLTLAS

SEQ ID NO: 21 - protein, 6 aa, Artificial Sequence
  APR
AGWFIA

SEQ ID NO: 22 - protein, 6 aa, Artificial Sequence
  APR
AGWFIA

SEQ ID NO: 23 - protein, 5 aa, Artificial Sequence
  APR
IIAAL

SEQ ID NO: 24 - protein, 7 aa, Artificial Sequence
  APR
GIIAALG

SEQ ID NO: 25 - protein, 7 aa, Artificial Sequence
  APR
IVVIYTT

SEQ ID NO: 26 - protein, 7 aa, Artificial Sequence
  APR
IVVIYTT

SEQ ID NO: 27 - protein, 21 aa, Artificial Sequence
  non-natural molecule
RFFAAFCLRRPRFFAAFCLRR

SEQ ID NO: 28 - protein, 21 aa, Artificial Sequence
  non-natural molecule
RLLLTTIGRRPRLLLTTIGRR

SEQ ID NO: 29 - protein, 19 aa, Artificial Sequence
  non-natural molecule
RLLTLASRRPRLLTLASRR

SEQ ID NO: 30 - protein, 19 aa, Artificial Sequence
  non-natural molecule
RAGWFIARRPRAGWFIARR

SEQ ID NO: 31 - protein, 21 aa, Artificial Sequence
  non-natural molecule
RGIIAALGRRPRGIIAALGRR

SEQ ID NO: 32 - protein, 21 aa, Artificial Sequence
  non-natural molecule
RIVVIYTTRRPRIVVIYTTRR

SEQ ID NO: 33 - protein, 4 aa, Artificial Sequence
  linker
GSGS

SEQ ID NO: 34 - protein, 7 aa, Artificial Sequence
  APR
SVIIWSL

SEQ ID NO: 35 - protein, 23 aa, Artificial Sequence
  non-natural molecule
RSVIIWSLGRRPRSVIIWSLGRR

SEQ ID NO: 36 - protein, 9 aa, Artificial Sequence
  linker
KPAGAAKGG

SEQ ID NO: 37 - protein, 9 aa, Artificial Sequence
  non-natural molecule
KELTAFLHN

SEQ ID NO: 38 - protein, 9 aa, Artificial Sequence
  non-natural molecule
AGLTAFLRQ

(19) United States
(12) Patent Application Publication
(10) Pub. No.: US 2024/0165196 A1
(43) Pub. Date: May 23, 2024

(54) Beta-Lactamase Inhibitors

(71) Applicants: VIB VZW, Gent (BE); KATHOLIEKE UNIVERSITEIT LEUVEN, Leuven (BE)

(72) Inventors: Joost Schymkowitz, Meensel-Kiezegem (BE); Frederic Rousseau, Groot-Bijgaarden (BE); Ladan Khodaparast, Bertem (BE); Laleh Khodaparast, Bertem (BE)

(21) Appl. No.: 18/548,706

(22) PCT Filed: Mar. 3, 2022
(86) PCT No.: PCT/EP2022/055387

§ 371 Date: Sep. 1, 2023

(30) Foreign Application Priority Data
Mar. 3, 2021 (EP) 21160529.0

(51) Int. Cl. A61K 38/16; A61K 31/43; A61K 38/08; A61P 31/04

(52) U.S. Cl. CPC A61K 38/16; A61K 31/43; A61K 38/08; A61P 31/04

(57) ABSTRACT

The present invention relates to the field of microbiology, in particular to the field of bacterial antibiotic resistance. more particularly to the field of resistance to beta-lactam inhibitors. The invention provides non-natural compounds which induce the aggregation of beta-lactamases, particularly beta-lactamases of class A. In addition, the invention provides combination therapies and pharmaceutical compositions between the non-natural molecules and beta-lactam antibiotics.

### CROSS REFERENCE TO RELATED APPLICATIONS

This application is a national phase entry under 35 U.S.C. § 371 of International Patent Application PCT/EP2022/055387, filed Mar. 3, 2022, designating the United States of America and published in English as International Patent Publication WO 2022/184821 on Sep. 9, 2022, which claims the benefit under Article 8 of the Patent Cooperation Treaty to European Patent Application Serial No. 21160529.0, filed Mar. 3, 2021, the entireties of which are hereby incorporated by reference.

### FIELD OF THE INVENTION

The present invention relates to the field of microbiology, in particular to the field of bacterial antibiotic resistance, more particularly to the field of resistance to beta-lactam inhibitors. The invention provides non-natural compounds which induce the aggregation of beta-lactamases, particularly beta-lactamases of class A. In addition, the invention provides combination therapies between the non-natural molecules and beta-lactam antibiotics.

### INTRODUCTION TO THE INVENTION

Bacterial resistance to penicillin was identified even before its introduction for therapeutic use¹ and is thought to have arisen from the selective pressures exerted by β-lactam producing soil organisms². Currently, Extended-Spectrum Beta-Lactamases (ESBLs) have evolved into widespread resistance factors that mediate bacterial tolerance of beta-lactam antibiotics by hydrolysis of the beta-lactam ring, including penicillins, cephalosporins, and to a lesser extent cephamycins and carbapenems³. The number of reported beta-lactamases continues to grow rapidly and currently exceeds over 200 different enzymes⁴, and for many of these there are 10s or 100s of closely related variants with an increased activity spectrum⁴. These enzymes are grouped into four amino acid sequence-based classes, of which A, C and D use a serine-mediated hydrolysis mechanism, whereas the mechanism of class B involves a divalent zinc ion⁵. A particular threat to global health care are the ESBLs with a resistance spectrum that now includes second-, third- and fourth-generation cephalosporins and monobactams. These strains are also resistant to current β-lactamase inhibitors², tazobactam, clavulanate and sulbactam, which are active-site competitors that form terminal covalent intermediates that inactivate the enzyme⁶. In gram negative bacteria, notably Escherichia coli (E. coli) and Klebsiella pneumoniae (K. pneumoniae), the biggest source of ESBLs are the class A beta-lactamases TEM and SHV, with over 500 variants of these enzymes recorded in the beta-lactamase database⁴. TEM-1 was the first plasmid-born β-lactamase identified in gram negatives, and was found in 1965 in an E. coli isolate from a patient in Athens, Greece, by the name of Temoneira⁷. Whereas TEM-1 conferred resistance to penicillin and early cephalosporins the enzyme has demonstrated a striking functional plasticity, adapting the active site to newer beta-lactam antibiotics that were specifically designed to withstand enzymatic hydrolysis. TEM beta-lactamases have spread worldwide and are found in different pathogens, including Enterobacteriaceae, Pseudomonas aeruginosa, Haemophilus influenzae, and Neisseria gonorrhoeae^{4,8}. SHV-1 (for Sulfhydryl variable) which shares 68% sequence identity with TEM-1, is chromosomally encoded by the vast majority of the resistant K. pneumoniae⁹.

Targeting the active site of beta-lactamases as a means of building inhibitors is an attractive idea since even structurally unrelated enzymes that target the same beta-lactam moiety could share some structural similarity in their active site, raising the possibility that a single inhibitor molecule might be effective against a range of target enzymes. Indeed, many beta-lactamase inhibitors, such as tazobactam, are so-called mechanism-based inhibitors that work by poisoning the active site with a non-hydrolysable substrate analog. One downside of this approach is the increased selective pressure this exerts on the already fast evolving beta-lactamase active site. A second, and perhaps less obvious potential downside of this approach is that substrate analogs are known to act as molecular chaperones¹⁰. Well-known example of such stabilizing substrate analogs in clinical use are deoxygalactonojirimycin (DGJ)^{11,12}, that is used as a molecular chaperone to rescue the folding of alpha-galactosidase in Fabry disease patients, and tafamidis, a molecular chaperone used for rescuing the folding of transthyretin in familial systemic amyloidosis patients¹³. In particular, the case of DGJ is relevant, since this molecule is an inhibitor that binds in the active site, but when administered at low doses it increases the overall activity of the enzyme by increasing the folding efficiency.

In the present invention we tested an orthogonal approach for inhibiting beta-lactamase activity, whereby inactivation of β-lactamases is achieved by induced protein misfolding using peptides that target aggregation-prone regions (APRs) specific to these enzymes. Importantly these APRs lie outside the active site and are generally conserved in current mutant versions of the same enzyme. Amyloid-like aggregation is an ordered process resulting in an aligned in-register packing of residues in amyloid assemblies. The ordered nature of amyloid-like assemblies also explains why aggregation can be catalysed by seeding, i.e. by the addition of a sub-stoichiometric amount of pre-formed aggregates, in a manner that is similar to the seeding of crystal growth¹⁴. Amyloid seeding is sequence-specific and even a single point mutation is often sufficient to impair seeding between homologues¹⁵. The vast majority of proteins in any proteome possess at least one aggregation prone region that can form amyloid-like aggregates. The ubiquity of APRs in proteins is a consequence of globular structure as its tertiary structure requires hydrophobic aggregation-prone sequence segments^{16,17}. Intriguingly, most APRs have a sequence that is unique in their proteome¹⁵. Based on this, the aggregation of any protein could in principle be specifically induced by seeding with a synthetic peptide encoding an APR of this target protein. As protein aggregation generally results in protein inactivation, this entails that APRs could therefore be exploited as sequence barcodes directing specific peptide-induced protein functional knockdown. We have recently tested its feasibility in diverse model systems, including prokaryotes^{18,19}, plants^{20,21} and mammalian cells²².

In the present invention we investigated whether synthetic amyloid-forming peptides could be developed that can induce the aggregation of the TEM and SHV beta-lactamases. In addition, we investigated whether the sensitivity to beta-lactam antibiotics in resistant clinical isolates could be restored with these artificial peptides. Peptides could be particularly suited as anti-bacterials since bacteria rely on the uptake of peptides as a source of amino acids, nitrogen and carbon³².

### SUMMARY OF THE INVENTION

In the present invention we have surprisingly shown that TEM and SHV beta-lactamases can be selectively aggregated by via targeted aggregation. The aggregation of TEM and SHV is in itself not a lethal event to the bacterial cells, but restores sensitivity to beta-lactam antibiotics, indicating that loss-of-function to aggregation is only toxic under conditions where the affected protein is essential for survival. The peptides show a striking selectivity between the analyzed beta-lactamases, as synergy is only observed between each specific pair of peptides and the enzyme it is targeting. The advantage is that identifying inhibitors for newly emerging enzymes is much faster than identifying novel small molecules.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A-1J. (FIG. 1A) Aggregation propensity prediction of TEM-1 using TANGO (left) and structural views of the TEM protein with the aggregation prone region predicted by TANGO highlighted (right). Structure image generated with Yasara of pdb ID 1bt5. (FIG. 1B) Distribution of the number of APRs per 100 residues in the various SCOP categories of protein folds: all-alpha helical (a), all beta-sheet (b), mixed helix and sheet (c) or separate helical and sheet segments (d). The dashed red line indicates the position of the TEM protein. (FIG. 1C) Structured illumination Microscopy (SIM) super-resolution image of E. coli BL21 overexpressing a GFP-fusion of TEM-1, showing a single representative micrograph of one out of three independent repeats performed. (FIG. 1D) Elution profile of recombinantly purified TEM-1 upon size-exclusion chromatography followed by Multi-Angle Light Scattering mass detection (SEC-MALS). Inset: Coomassie-stained SDS-PAGE of the same sample. The continuous green line is the UV signal (right Y-axis), the dots near the center of the peak are the molecular masses measured in parallel (left Y-axis). The data show single representative run of one of three independent repeats. (FIG. 1E) Heat denaturation of TEM-1 monitored by intrinsic fluorescence plotted as the BaryCentric Mean (BCM) of the fluorescence emission spectrum in the presence (red) and absence (blue) of tazobactam. The melting temperature (Tm) is derived from these data. The plot shows the mean of 7 replicates, and the error bars represent the standard deviation. (FIG. 1F) Temperature-dependent evolution of the Right-Angle Light Scattering (RALS) intensity measured simultaneously with the data in FIG. 1G to monitor protein aggregation. The aggregation onset temperature Tagg is derived from these data. The plot shows the mean of 7 replicates, and the error bars represent the standard deviation. (FIGS. 1G, 1H, 1I, and 1J) Same data as in FIG. 1F, but showing a dose-titration of different beta-lactamase inhibitors: tazobactam (FIG. 1G), clavulanate (FIG. 1H), vaborbactam (FIG. 1I) and avibactam (FIG. 1J). Each curve a single experiment, which was repeated in triplicate.

FIGS. 2A-2M. (FIG. 2A) TEM mutations and their effects on stability (total energy) as predicted by FoldX. Mutations are categorized according to whether they have been shown to afford resistance to β-lactam antibiotics (“BLACT_RES”), or inhibitors (“INH_RES”), or whether they stabilize protein structure (“stability”). Dashed line indicates a total energy difference of 0.5 kcal/mol, the FoldX cut-off above which mutations are considered to be destabilizing. (FIG. 2B) Barplot mapping mutations in (FIG. 2A) to the TEM sequence and indicating occurrence. X-axis shows the position in the primary TEM sequence. The bar heights correspond to the number of times a mutation occurs across different TEM variants (right-hand y-axis). The color coding is identical to that in (FIG. 2A). Red line indicates the TANGO scores (indicated on the left-hand y-axis), with peaks corresponding to APRs. (FIG. 2C) Mapping of mutations in (FIG. 2A) and (FIG. 2B) to the TEM structure (pdb 1xpb). Protein surface is shown in gray, color coding of mutations is identical to (FIG. 2A) and (FIG. 2B). (FIGS. 2D, 2E, and 2F) Temperature-dependent evolution of the Right-Angle Light Scattering (RALS) intensity during a temperature ramp, similar as in FIG. 1F for key mutants of TEM1, namely TEM-10 (FIG. 2D), TEM-30 (FIG. 2E) and TEM-155 (FIG. 2F). Experiments were shown in triplicate, single repeat is shown. (FIG. 2G) Schematic representation of the structure of the peptides. APR—Aggregation Prone Region, R—Arginine. (FIG. 2H) Ribbon representation of the superposition of the crystal structures of TEM (green, pdb id 1bt5) and SHV (blue, pdb id 1shv). The catalytic site of the beta-lactamase activity is indicated and location of APR3 is shown in red. (FIG. 2I) TANGO aggregation score and alignment of APR3 in TEM and SHV. (FIG. 2J) Fractional Inhibitory Concentration Index determination of 5 peptides versus penicillin on E. coli TEM-104. The plot shows all data recorded; Each dot indicates an independent repeat of the measurement consisting of 96 datapoints. FICI values below 0.5 indicate synergy. Values between 0.5 and 1.0 indicate additivity and values greater than 1 indicate indifference between the combined substances. (FIG. 2K) Same as J for E. coli SHV-11. (FIG. 2L) Same as J for a kanamycin resistant E. coli strain. (FIG. 2M) FICI values for the TEM3.2 peptide on a range of E. coli strains.

FIGS. 3A-3P. (FIG. 3A) Measurements of the hydrodynamic radius by Dynamic Light Scattering of 50 μM TEM3.2 in buffer alone (50 mM Tris pH 8.5, 300 mM NaCl), or in the presence of LPS or polyphosphate (PolyP). The data show a single representative replicate. (FIG. 3B) Amyloid-like aggregation kinetics of 50 μM TEM3.2 measured by Thioflavin-T (Th-T) fluorescence in the same conditions as FIG. 3A. Three replicates are shown for each condition. (FIG. 3C) Transmission Electron Micrograph of 50 μM TEM 3.2 incubated for 24 h in the same buffer as FIG. 3A with polyP, negatively stained with 2% (w/v) uranyl acetate. A single representative image is shown. (FIG. 3D) Amyloid-like aggregation kinetics of 50 μM TEM 3.2 in the PolyP condition in FIG. 3A, measured by pFTAA fluorescence. Three replicates are shown for each condition. (FIG. 3E) [Left] Aggregation kinetics using pFTAA fluorescence emission of recombinantly purified TEM-1, in the presence of vehicle, or TEM 3.2 peptin, as well as peptin-only control. [Middle] Similar as in the left panel, but no using an off-target Pept-In, based on an APR sequence of beta-galactosidase. [Right] Similar as in the left panel, but now using a previously published off-target Pept-In, based on an APR of HcaB. In all panels, the average of 2 replicates are shown and the error bars represent the standard error of the em deviation. (FIG. 3F) Structured Illumination Microscopy (SIM) image, side by chain with a brightfield image of E. coli strain UZ_TEM104 treated with TEM3.2 at 12 μM for 120 min and stained with pFTAA. A single representative image is shown. (FIG. 3G) SIM image of E. coli strain UZ_TEM104 treated with 12 μM FITC-TEM 3.2 in PBS for 120 min, and stained with the red-shifted oligothiophene HS169 to visualise the aggregation. A single representative image is shown. (FIG. 3H) SIM and brightfield image as in FIG. 3F, but for E. coli ATCC strain. (FIG. 3I) SIM images of E. coli K12 MG1655 overexpressing GFP from a pBAD vector. A single representative image is shown. (FIG. 3J) Western blot and quantification for the TEM beta-lactamase in the Inclusion Body (IB) fraction of E. coli strain UZ_TEM104, treated with 12 UM of the indicated peptide in PBS for 120 min or control. A single representative blot is shown. The quantification is the result of the densitometric quantification of 4 independent replicate blots and show the mean and the standard deviation. (FIG. 3K) Western blot and quantification for the SHV beta-lactamase in the IB fraction of E. coli SHV-11, treated with 12 UM of the indicated peptide in PBS for 120 min or control. (FIG. 3L) Fluorescence Activated Cell-Sorting (FACS) of E. coli strain UZ_TEM104 mixed 50-50% with the same strain after heat-inactivation. The cells were stained with pFTAA to monitor aggregation and Propidium Iodide (PI) to monitor cell permeabilization associated to cell death. 106 cells were analysed for the plot. The plot shows a single representative run of three independent repeats. (FIG. 3M) Similar FACS analysis as in FIG. 3L, but with a sample of live bacteria only. (FIG. 3N) Similar FACS analysis as in FIG. 3L, but for the same E. coli strain treated for 4 h with 400 μg/mL penicillin. (FIG. 3O) Similar FACS analysis as in FIG. 3L, but treated with 50 μg/mL TEM 3.2 in PBS for 4 h. (FIG. 3P) Similar FACS analysis as in FIG. 3L, but treated with 400 μg/mL penicillin and 50 μg/mL TEM3.2 in PBS for 4 h.

FIGS. 4A-4K. (FIG. 4A) Lysis of human erythrocytes (hemolysis) treated with the indicated concentrations of TEM3.2 for 2 h at 37° C., normalized to the value obtained with 1% of the detergent triton. The plot is the result of 3 replicates and shows the mean and the standard deviation. (FIG. 4B) Cell viability using the CellTiter Blue assay of Hela cells treated for 24 h with the indicated concentrations TEM 3.2 at 37° C. The plot is the result of 3 replicates and shows the mean and the standard deviation. (FIG. 4C) co-cultures of human cell lines and E. coli TEM1 with a FITC-labelled derivative of peptide TEM3.2, fluorescence is only observed in the bacterial but not the mammalian cells indicating preferential uptake into the bacteria, (FIGS. 4D, 4E, and 4F) Bacterial load of indicated organs of female C57BL/6JAX mice with a urinary tract infection with E. coli strain UZ_TEM104, treated with 30 mg/kg penicillin (oral) as well 10 mg/kg tazobactam or 10 mg/kg TEM3.2 via the indicated treatment route. IV—Intravenous, IP—Intraperitoneal, SC—Subcutaneously. The data shown is from a single experiment with 4 animals per group. The result for each animal is shown as a dot. The plot also shows a box plot, where the box extends from the 25th to 75th percentiles, the line in the middle of the box is plotted at the median and the whiskers go from the minimum to maximum values. The statistically significant differences were determined using ANOVA, followed by Dunnett's multiple comparison test. (ns—non significant, #—P>0.05, *—P≤0.05, ***—P≤0.01, ****—P≤0.001). (FIG. 4G) FICI plot as in FIG. 2, for the E. coli strains indicated and the combination of penicillin and the TEM3.2 peptide. (FIG. 4H) FICI plot as in FIG. 2, for the strains indicated treated with penicillin and peptide NDM1-1. (FIG. 4I) Same as FIG. 4H for peptide NDM1-2. (FIG. 4J) Same as FIG. 4H for peptide BGAL. (FIG. 4K) Same as FIG. 4H for peptide P33.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The introduction of new antibiotics and the development of resistance against these molecules by the bacteria that are their target constitutes a global arms race that will continue for as long as novel antibiotics are introduced⁴⁹. However, most antibiotics can be classified into a few chemical families and the need for truly novel chemical classes of antibiotics was dramatically illustrated by the reduced time to resistance of more recently developed molecules, with resistance mechanisms to very similar molecules probably being present in the population prior to market release. The development of non-hydrolysable substrate analogs of beta-lactamases as drugs to inhibit that particular class of resistance factors could be seen in the same light: by increasing the selective pressure the beta-lactamase active site is under, these drugs may lead to the perverse effects of accelerating beta-lactamase evolution. Therefore, a need exists for alternative strategies and we here explored the potential of targeted protein aggregation for this purpose.

As corroborated by the experimental section, which illustrates certain representative embodiments of the present invention, the inventors for the first time disclose and demonstrate the therapeutic potential of molecules which comprise one or more β-aggregating sequences designed to specifically target β-aggregation prone regions (APRs) that arise in extended-spectrum beta-lactamase (ESBL), more specifically in ESBL of class A.

Accordingly, an aspect provides a non-naturally occurring molecule configured to form an intermolecular beta-sheet with a bacterial ESBL protein, in particular an ESBL protein of class A.

In a particular embodiment the invention provides a non-naturally occurring molecule configured to form an intermolecular beta-sheet with an extended-spectrum beta-lactamase of class A wherein the molecule comprises, consists essentially of or consists of the structure:
a. NGK1-P1-CGK1,
b. NGK1-P1-CGK1-Z1-NGK2-P2-CGK2, or
c. NGK1-P1-CGK1-Z1-NGK2-P2-CGK2-Z2-NGK3-P3-CGK3,
   wherein:
   P1 to P3 each independently denote an amino acid stretch comprising

i)
(SEQ ID NO: 1)
LTAFLX₁X₂
wherein X₁ is H or R
and
X₂ is N or Q,
or
ii)
(SEQ ID NO: 2)
TAQILNW,
or
iii)
(SEQ ID NO: 3)
AQILNWI,
or
iv)
(SEQ ID NO: 4)
LAAALML

NGK1, NGK2, NGK3; CGK1, CGK2 and CGK3 each independently denote 1 to 3 contiguous amino acids that display low beta-sheet potential or a propensity to disrupt beta-sheets, preferably 1 to 3 contiguous amino acids selected from the group consisting of R, K, E, D and P, D-isomers and/or analogues thereof, and combinations thereof, and
Z1 and Z2 each independently denote a linker.

In a particular embodiment each linker in the non-natural molecule is independently selected from a stretch between 1 and 5 units, wherein a unit is independently an amino acid or PEG, such as wherein each linker is independently GS, P, PP, or D-isomers and/or analogues thereof.

In another particular embodiment the non-natural molecule comprises, consists essentially of or consists of a peptide of the amino acid sequence:

a.
(SEQ ID NO: 5)
RLTAFLHNRRPRLTAFLHNRR,
or
b.
(SEQ ID NO: 6)
RLTAFLRQRRPRLTAFLRQRR,
or
c.
(SEQ ID NO: 7)
RLTAFLHNRRPRLTAFLRQRR,
or
d.
(SEQ ID NO: 8)
RLTAFLRQRRPRLTAFLHNRR,

optionally wherein the amino acid sequence comprises one or more D-amino acids and/or analogues of one or more of its amino acids, optionally wherein the N-terminal amino acid is acetylated and/or the C-terminal amino acid is amidated.

In yet another embodiment the invention provides a non-naturally occurring molecule configured to form an intermolecular beta-sheet with an extended-spectrum beta-lactamase of class A wherein the intermolecular beta-sheet involves:
a. a portion of or the whole of the amino acid sequence LTAFLHN (SEQ ID NO: 9) present in the extended-spectrum beta-lactamase protein of class A and/or
b. a portion of or the whole of the amino acid sequence LTAFLRQ (SEQ ID NO: 10) present in the extended-spectrum beta-lactamase protein of class A.

In a particular embodiment in the non-natural molecule the amino acid stretch comprises one or more D-amino acids and/or analogues of one or more of its amino acids.

In yet another embodiment the non-natural molecules of the invention comprise a detectable label, a moiety that allows for isolation of the molecule, a moiety increasing the stability or half-life of the molecule, a moiety increasing the solubility of the molecule, and/or a moiety increasing the bacterial uptake of the molecule.

In yet another embodiment the invention provides the combination of a non-naturally occurring molecule as herein before described and a beta-lactam antibiotic such as penicillin derivatives, cephems, penems, monobactams, clavams, carbacephems or oxacephems.

In yet another embodiment the invention provides a non-naturally occurring molecule as herein described for use in medicine.

In yet another embodiment the invention provides the combination of a non-naturally molecule as herein described and a beta-lactam antibiotic such as penicillin derivatives, cephems, penems, monobactams, clavams, carbacephems or oxacephems for use in medicine.

In yet another embodiment the invention provides a non-naturally occurring molecule as herein described for use to treat a bacterial infection.

In yet another embodiment the invention provides the combination of a non-naturally molecule as herein described and a beta-lactam antibiotic such as penicillin derivatives, cephems, penems, monobactams, clavams, carbacephems or oxacephems for use to treat a bacterial infection.

In yet another embodiment the invention provides the combination of a non-naturally molecule as herein described and a beta-lactam antibiotic such as penicillin derivatives, cephems, penems, monobactams, clavams, carbacephems or oxacephems for use to treat a bacterial infection and optionally with a further molecule which is a beta-lactam inhibitor.

In yet another embodiment the invention provides a pharmaceutical composition comprising a non-naturally occurring molecule as herein described.

In yet another embodiment the invention provides a pharmaceutical composition comprising a combination or a kit of parts of a non-naturally occurring molecule as herein described and a beta-lactam antibiotic such as penicillin derivatives, cephems, penems, monobactams, clavams, carbacephems or oxacephems for use to treat a bacterial infection and optionally with a further molecule which is a beta-lactam inhibitor.

The term “non-naturally occurring” generally refers to a material or an entity that is not formed by nature or does not exist in nature. Such non-naturally occurring material or entity may be made, synthesised, semi-synthesised, modified, intervened on or manipulated by man using methods described herein or known in the art. By means of an example, the term when used in relation to a peptide may in particular denote that a peptide of an identical amino acid sequence is not found in nature, or if a peptide of an identical amino acid sequence is present in nature, that the non-naturally occurring peptide comprises one or more additional structural elements such as chemical bonds, modifications or moieties which are not included in and thus distinguish the non-naturally occurring peptide from the naturally occurring counterpart. In certain embodiments, the term when used in relation to a peptide may denote that the amino acid sequence of the non-naturally occurring peptide is not identical to a stretch of contiguous amino acids encompassed by a naturally occurring peptide, polypeptide or protein. For avoidance of doubt, a non-naturally occurring peptide may perfectly contain an amino acid stretch shorter than the whole peptide, wherein the structure of the amino acid stretch including in particular its sequence is identical to a stretch of contiguous amino acids found in a naturally occurring peptide, polypeptide or protein.

In the context of the present disclosure, the phrase “a molecule configured to” intends to encompass any molecule that exhibits the recited outcome or functionality under appropriate circumstances. Hence, the phrase can be seen as synonymous to and interchangeable with phrases such as “a molecule suitable for”, “a molecule having the capacity to”, “a molecule designed to”, “a molecule adapted to”, “a molecule made to”, or “a molecule capable of”.

The terms “beta-sheet”, “beta-pleated sheet”, “β-sheet”, “β-pleated sheet” are well-known in the art and by virtue of additional explanation interchangeably refer to a molecular structure comprising two or more beta-strands connected laterally by backbone hydrogen bonds (inter-strand hydrogen bonding). A beta-strand is a stretch of amino acids typically 3 to 10 amino acids long with backbone in an almost fully extended conformation, following a ‘zigzag’ trajectory. Adjacent amino acid chains in a beta-sheet can run in opposite directions (antiparallel β sheet) or in the same direction (parallel β sheet) or may show a mixed arrangement. When not forming a beta-sheet (e.g., prior to participating in a beta-sheet), the stretch of amino acids may exhibit a non-beta-strand conformation; for example it may have an unstructured conformation.

An “intermolecular” beta-sheet involves beta-strands from two or more separate molecules, such as from two or more separate peptides or peptide-containing molecules, polypeptides and/or proteins. In the context of the instant disclosure, the term particularly denotes a beta-sheet involving one or more beta-strands from one or more molecules as taught herein and one or more beta-strands from one or more ESBL molecules. Given that co-aggregation seeded by the intermolecular beta-sheet formation is considered to play an important role in the mode of action of the present molecules, many tens, hundreds, thousands, or more molecules as taught herein and molecules of ESBL proteins may be involved in underlying beta-sheets interactions, leading to higher order organisation and structures, such as protofibrils, fibrils and aggregates.

Typically, a beta-strand may be formed by only a part of (e.g., by a stretch of contiguous amino acids of) a molecule, peptide, polypeptide or protein that participates in a beta-sheet. For example, the molecule as taught herein may include one or more stretches of contiguous amino acids which become organised into beta-strands participating in beta-sheets in cooperation with one or more beta-strands constituted by stretches of contiguous amino acids of one or more ESBL protein molecules. In other words, a statement that a molecule can form and intermolecular beta-sheet with a bacterial ESBL protein will typically mean that one or more portions of the molecule, such as one or more stretches of contiguous amino acids of the molecule, is or are designed to organise into beta-strands that can participate in a beta-sheet together with one or more stretches of contiguous amino acids of a bacterial ESBL molecule. The interlocking of beta-strands from two or more separate molecules into beta sheets can thus create a complex in which the two or more separate molecules become physically associated or connected and spatially adjacent. In view of the aforementioned explanations, the phrase “a molecule configured to form an intermolecular beta-sheet with a bacterial ESBL protein” may also subsume the meanings: a molecule capable of participating in or contributing to or inducing the generation of an intermolecular beta-sheet with a stretch of contiguous amino acids of a bacterial ESBL protein; a molecule comprising a portion capable of participating in or contributing to or inducing the generation of an intermolecular beta-sheet with a stretch of contiguous amino acids of a bacterial ESBL RAS protein; and a molecule comprising a stretch of contiguous amino acids capable of participating in or contributing to or inducing the generation of an intermolecular beta-sheet with a stretch of contiguous amino acids of a bacterial ESBL protein.

The term “protein” generally encompasses macromolecules comprising one or more polypeptide chains. The term “polypeptide” generally encompasses linear polymeric chains of amino acid residues linked by peptide bonds. A “peptide bond”, “peptide link” or “amide bond” is a covalent bond formed between two amino acids when the carboxyl group of one amino acid reacts with the amino group of the other amino acid, thereby releasing a molecule of water. Especially when a protein is only composed of a single polypeptide chain, the terms “protein” and “polypeptide” may be used interchangeably to denote such a protein. The terms are not limited to any minimum length of the polypeptide chain. Polypeptide chains consisting essentially of or consisting of 50 or less (≤50) amino acids, such as ≤45, ≤40, ≤35, ≤30, ≤25, ≤20, ≤15, ≤10 or ≤5 amino acids may be commonly denoted as a “peptide”. In the context of proteins, polypeptides or peptides, a “sequence” is the order of amino acids in the chain in an amino to carboxyl terminal direction in which residues that neighbour each other in the sequence are contiguous in the primary structure of the protein, polypeptide or peptide. The terms may encompass naturally, recombinantly, semi-synthetically or synthetically produced proteins, polypeptides or peptides. Hence, for example, a protein, polypeptide or peptide can be present in or isolated from nature, e.g., produced or expressed natively or endogenously by a cell or tissue and optionally isolated therefrom; or a protein, polypeptide or peptide can be recombinant, i.e., produced by recombinant DNA technology, and/or can be, partly or entirely, chemically or biochemically synthesised. Without limitation, a protein, polypeptide or peptide can be produced recombinantly by a suitable host or host cell expression system and optionally isolated therefrom (e.g., a suitable bacterial, yeast, fungal, plant or animal host or host cell expression system), or produced recombinantly by cell-free translation or cell-free transcription and translation, or non-biological peptide, polypeptide or protein synthesis. The terms also encompasses proteins, polypeptides or peptides that carry one or more co- or post-expression-type modifications of the polypeptide chain(s), such as, without limitation, glycosylation, lipidation, acetylation, amidation, phosphorylation, sulphonation, methylation, pegylation (covalent attachment of polyethylene glycol typically to the N-terminus or to the side-chain of one or more Lys residues), ubiquitination, sumoylation, cysteinylation, glutathionylation, oxidation of methionine to methionine sulphoxide or methionine sulphone, signal peptide removal, N-terminal Met removal, conversion of pro-enzymes or pre-hormones into active forms, etc. Such co- or post-expression-type modifications may be introduced in vivo by a host cell expressing the proteins, polypeptides or peptides (co- or post-translational protein modification machinery may be native to the host cell and/or the host cell may be genetically engineered to comprise one or more (additional) co- or post-translational protein modification functionalities), or may be introduced in vitro by chemical (e.g., pegylation) and/or biochemical (e.g., enzymatic) modification of the isolated proteins, polypeptides or peptides.

The term “amino acid” encompasses naturally occurring amino acids, naturally encoded amino acids, non-naturally encoded amino acids, non-naturally occurring amino acids, amino acid analogues and amino acid mimetics that function in a manner similar to the naturally occurring amino acids, all in their D- and L-stereoisomers, provided their structure allows such stereoisomeric forms. Amino acids are referred to herein by either their name, their commonly known three letter symbols or by the one-letter symbols recommended by the IUPAC-IUB Biochemical Nomenclature Commission. A “naturally encoded amino acid” refers to an amino acid that is one of the 20 common amino acids or pyrrolysine, pyrroline-carboxy-lysine or selenocysteine. The 20 common amino acids are: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). A “non-naturally encoded amino acid” refers to an amino acid that is not one of the 20 common amino acids or pyrrolysine, pyrroline-carboxy-lysine or selenocysteine. The term includes without limitation amino acids that occur by a modification (such as a post-translational modification) of a naturally encoded amino acid, but are not themselves naturally incorporated into a growing polypeptide chain by the translation complex, as exemplified without limitation by N-acetylglucosaminyl-L-serine, N-acetylglucosaminyl-L-threonine, and O-phosphotyrosine. Further examples of non-naturally encoded, un-natural or modified amino acids include 2-Aminoadipic acid, 3-Aminoadipic acid, beta-Alanine, beta-Aminopropionic acid, 2-Aminobutyric acid, 4-Aminobutyric acid, piperidinic acid, 6-Aminocaproic acid, 2-Aminoheptanoic acid, 2-Aminoisobutyric acid, 3-Aminoisobutyric acid, 2-Aminopimelic acid, 2,4 Diaminobutyric acid, Desmosine, 2,2′-Diaminopimelic acid, 2,3-Diaminopropionic acid, N-Ethylglycine, N-Ethylasparagine, homoserine, homocysteine, Hydroxylysine, allo-Hydroxylysine, 3-Hydroxyproline, 4-Hydroxyproline, Isodesmosine, allo-Isoleucine, N-Methylglycine, N-Methylisoleucine, 6-N-Methyllysine, N-Methylvaline, Norvaline, Norleucine, or Ornithine. Also included are amino acid analogues, in which one or more individual atoms have been replaced either with a different atom, an isotope of the same atom, or with a different functional group. Also included are un-natural amino acids and amino acid analogues described in Ellman et al. Methods Enzymol. 1991, vol. 202, 301-36. The incorporation of non-natural amino acids into proteins, polypeptides or peptides may be advantageous in a number of different ways. For example, D-amino acid-containing proteins, polypeptides or peptides exhibit increased stability in vitro or in vivo compared to L-amino acid-containing counterparts. More specifically, D-amino acid-containing proteins, polypeptides or peptides may be more resistant to endogenous peptidases and proteases, thereby providing improved bioavailability of the molecule and prolonged lifetimes in vivo.

The characterisation of the present molecules as being able to form an intermolecular beta-sheet with bacterial ESBL proteins is based inter alia on the mechanisms described in WO 2007/071789A1 and WO2012/123419A1 as underlying the operation of the ‘interferor’ technology. However, the emergence of beta-sheet conformation may also be experimentally assessed by available methods. By means of a non-limiting example, nuclear magnetic resonance (NMR) spectroscopy has been employed for many years to characterise the secondary structure of proteins in solution (reviewed in Wuetrich et al. FEBS Letters. 1991, vol. 285, 237-247).

Perhaps more straightforwardly in the context of the present invention, the formation of the intermolecular beta-sheet leads to an interaction between the non-natural molecule and the bacterial ESBL protein, which can be qualitatively and quantitatively assessed by standard methods such as co-immunoprecipitation assays, standard immunoassay or standard fluorescence microscopy methods.

As stated earlier, beta-strands tend to be 3 to 10 amino acids long. Accordingly, in certain embodiments the intermolecular beta-sheet formed between the molecule and its bacterial ESBL target may involve at least 3, such as at least 4 or at least 5, contiguous amino acids of the APR predicted in the bacterial ESBL protein.

Any meaningful extent of downregulation of the activity of the bacterial ESBL protein is envisaged. Hence, the terms “downregulate” or “downregulated”, or “reduce” or “reduced”, or “decrease” or “decreased” may in appropriate contexts, such as in experimental or therapeutic contexts, denote a statistically significant decrease relative to a reference. The skilled person is able to select such a reference. An example of a suitable reference may be the bacterial ESBL protein when exposed to a ‘negative control’ molecule, such as a molecule of similar composition but known to have no effects on the bacterial ESBL protein.

Any meaningful extent of reduction in solubility of the bacterial ESBL protein is envisaged. This may in appropriate contexts, such as in experimental or therapeutic contexts, denote a statistically significant decrease of the amount of bacterial ESBL protein present in the soluble protein fraction, or a statistically significant increase of the amount of bacterial ESBL protein present in the insoluble protein fraction, or a statistically significant decrease in the relative abundance of bacterial ESBL protein in the soluble vs. insoluble protein fractions, relative to a respective reference. The skilled person is able to select such a reference, such as in particular a reference indicative of bacterial ESBL solubility in the presence of a ‘negative control’ molecule.

The present molecules are able to induce the formation of an intermolecular beta-sheet with a bacterial ESBL protein. To this end, the molecules may advantageously comprise at least one portion that can assume or mimic a beta-strand conformation capable of interacting with the beta-strand contributed by the bacterial ESBL protein APR so as to give rise to an intermolecular beta-sheet formed by said interacting beta-strands.

As explained earlier, beta-strands tend to be 3 to 10 amino acids long. Accordingly, in certain embodiments the at least one amino acid stretch comprised by the molecule may be at least 3, such as at least 4 or at least 5, contiguous amino acids long. To enhance specificity of the interaction, the at least one amino acid stretch comprised by the molecule may be at least 6, such as exactly 6, or at least 7, such as exactly 7, or at least 8, such as exactly 8, or at least 9, such as exactly 9, or at least 10, such as exactly 10, contiguous amino acids long. Amino acid stretches that are 11, 12, 13 or 14 contiguous amino acids long can also be conceivably comprised by the molecule, but stretches of 6 to 10 contiguous amino acids may be preferred, since they allow for satisfactory specificity while simplifying the design of the molecules.

In certain preferred embodiments, the at least one stretch of amino acids, such as the at least one stretch of 6 to 10 contiguous amino acids, comprised by the molecule (henceforth “the ESBL molecule stretch” for brevity) may correspond to the stretch of contiguous amino acids within the APR of the bacterial ESBL protein which is to participate in the beta-sheet. By means of certain examples, when the beta-sheet is to involve a bacterial ESBL stretch of 3, 4, 5, preferably 6 to 10, such as 6, 7, 8, 9 or 10, or even 11, 12, 13 or 14 contiguous amino acids of the APR present in the bacterial APR, the molecule stretch can correspond to this bacterial ESBL stretch.

Further, as illustrated above, the molecule stretch, i.e., the at least 3, such as at least 4, at least 5 amino acids stretch comprised by the molecules as taught herein which participates in the intermolecular beta-sheet, may also include D-amino acids and/or analogues of the recited amino acids. Stated more generally, in certain embodiments, the at least one amino acid stretch of the molecule may comprise one or more D-amino acids, or analogues of one or more of its amino acids, or one or more D-amino acids and analogues of one or more of its amino acids, provided the incorporation of the D-amino acid or D-amino acids and/or the analogue or analogues is compatible with the formation of the intermolecular beta-sheet as taught herein.

Without limitation, in certain embodiments the molecule stretch may include only one D-amino acid. In certain embodiments, the molecule stretch may include two or more (e.g., 3, 4, 5, 6 or more) D-amino acids. In certain embodiments, about 10%, about 20%, about 30%, about 40%, about 50%, about 60%, about 70%, about 80%, about 90%, or 100% (i.e., all) amino acids constituting the molecule stretch may be D-amino acids. In certain embodiments, the D-amino acids may be interspersed between L-amino acids and/or the D-amino acids may be organised into one or more sub-stretches of two or more D-amino acids separated by L-amino acids. Without limitation, in certain embodiments the molecule stretch may include an analogue of only one of its amino acids. In certain embodiments, the molecule stretch may include analogues of two or more (e.g., 3, 4, 5, 6 or more) of its amino acids. In certain embodiments, the molecule stretch may include analogues of about 10%, about 20%, about 30%, about 40%, about 50%, about 60%, about 70%, about 80%, about 90%, or 100% (i.e., all) of its amino acids. In certain embodiments, the amino acid analogues may be interspersed between naturally occurring amino acids and/or the amino acid analogues may be organised into one or more sub-stretches of two or more such analogues separated by naturally occurring amino acids. Without limitation, in certain embodiments the molecule stretch may include only one constituent that is a D-amino acid or a amino acid analogue. In certain embodiments, the molecule stretch may include two or more (e.g., 3, 4, 5, 6 or more) constituents that are D-amino acids or amino acid analogues. In certain embodiments, about 10%, about 20%, about 30%, about 40%, about 50%, about 60%, about 70%, about 80%, about 90%, or 100% (i.e., all) constituents of the molecule stretch may be D-amino acids or amino acid analogues.

The reference to an amino acid analogue may encompass any compound that has the same or similar basic chemical structure as a naturally-encoded amino acid, i.e., an organic compound comprising a carboxyl group, an amino group, and an R moiety (amino acid residue). Typically, the amino group and the R moiety may be bound to the α carbon atom (i.e., the carbon atom to which the carboxyl group is bound). In other embodiments, the amino group may be bound to a carbon atom other than the α carbon atom, for example, to the β or γ carbon atom, preferably to the β carbon atom. In such embodiments, the R moiety may be bound to the same carbon atom as the amino group or to a carbon atom closer to the α carbon atom or to the α carbon atom itself. Typically, where the carboxyl group, the amino group and the R moiety are bound to the α carbon atom, the α carbon atom may also be bound to a hydrogen atom. Typically, where the amino group and the R moiety are bound to the β carbon atom, the β carbon atom may also be bound to a hydrogen atom. Without limitation, the R moiety of an amino acid analogue may differ from the R group of the respective naturally-encoded amino acid by one or more individual atoms or functional groups of the R group being replaced or substituted with a different atom (e.g., a methyl group replaced with a hydrogen atom, or an S atom replaced with an O atom, etc.), with an isotope of the same atom (e.g., ¹²C replaced with ¹³C, ¹⁴N replaced with ¹⁵N, or ¹H replaced with ²H, etc.), or with a different functional group (e.g., a hydrogen atom replaced with a methyl, ethyl or propyl group, or with another alkyl, alkenyl, cycloalkyl, cycloalkenyl, heterocyclyl, aryl, or heteroaryl group; an —SH group replaced with an —OH group or —NH₂ group, etc.). The structural difference or modification in an amino acid analogue compared to the respective naturally-encoded amino acid preferably preserves the core property of the amino acid with respect to charge and polarity. Hence, an amino acid analogue of a non-polar hydrophobic amino acid may preferably also have a non-polar hydrophobic R moiety; an amino acid analogue of a polar neutral amino acid may preferably also have a polar neutral R moiety; an amino acid analogue of a positively charged (basic) amino acid may preferably also have a positively charged R moiety, preferably with the same number of charged groups; and an amino acid analogue of a negatively charged (acidic) amino acid may preferably also have negatively charged R moiety, preferably with the same number of charged groups. All amino acid analogues are envisaged as both D- and L-stereoisomers, provided their structure allows such stereoisomeric forms.

By means of an example and without limitation, a leucine analogue may be selected from the list consisting of 2-amino-3,3-dimethyl-butyric acid (t-Leucine), alpha-methylleucine, hydroxyleucine, 2,3-dehydro-leucine, N-alpha-methyl-leucine, 2-Amino-5-methyl-hexanoic acid (homoleucine), 3-Amino-5-methylhexanoic acid (beta-homoleucine), 2-Amino-4,4-dimethyl-pentanoic acid (4-methyl-leucine, neopentylglycine), 4,5-dehydro-norleucine, L-norleucine, N-alpha-methyl-norleucine, and 6-hydroxy-norleucine, including their D- and L-stereoisomers, provided their structure allows such stereoisomeric forms. By means of an example and without limitation, a valine analogue may be selected from the list consisting of c-alpha-methyl-valine (2,3-dimethylbutanoic acid), 2,3-dehydro-valine, 3,4-dehydro-valine, 3-methyl-L-isovaline (methylvaline), 2-amino-3-hydroxy-3-methylbutanoic acid (hydroxyvaline), beta-homovaline, and N-alpha-methyl-valine, including their D-and L-stereoisomers, provided their structure allows such stereoisomeric forms. By means of an example and without limitation, a glycine analogue may be selected from the list consisting of N-alpha-methyl-glycine (sarcosine), cyclopropylglycine, and cyclopentylglycine, including their D- and L-stereoisomers, provided their structure allows such stereoisomeric forms. By means of an example and without limitation, an alanine analogue may be selected from the list consisting of 2-amino-isobutyric acid (2-methylalanine), 2-amino-2-methylbutanoic acid (isovaline), N-alpha-methyl-alanine, c-alpha-methyl-alanine, c-alpha-ethyl-alanine, 2-amino-2-methylpent-4-enoic acid (alpha-allylalanine), beta-homoalanine, 2-indanyl-glycine, di-n-propyl-glycine, di-n-butyl-glycine, diethylglycine, (1-naphthyl)alanine, (2-naphthyl)alanine, cyclohexylglycine, cyclopropylglycine, cyclopentylglycine, adamantyl-glycine, and beta-homoallylglycine, including their D- and L-stereoisomers, provided their structure allows such stereoisomeric forms.

In certain embodiments, the molecule may comprise exactly one amino acid stretch which participates in the intermolecular beta-sheet (i.e., exactly one ‘molecule stretch’ as discussed above). In certain preferred embodiments, the molecule may comprise two or more amino acid stretches which participate in the intermolecular beta-sheet (i.e., two or more ‘molecule stretches’ as discussed above). For example, the molecule may comprise 2 to 6, preferably 2 to 5, more preferably 2 to 4, or even more preferably 2 or 3 molecule stretches. For example, the molecule may comprise exactly 2, or exactly 3, or exactly 4, or exactly 5 molecule stretches, particularly preferably exactly 2 or exactly 3 molecule stretches, even more preferably exactly 2 molecule stretches. The inclusion of two or more molecule stretches tends to increase the effectiveness of the molecules in downregulating and inducing aggregation of bacterial ESBL proteins.

Where the molecule comprises two or more molecule stretches as taught herein, these may each independently be identical or different. For example, in a molecule with exactly 2 molecule stretches, the 2 molecule stretches may be identical or different; in a molecule with exactly 3 molecule stretches, all 3 stretches may be identical, or each stretch may be different from each other stretch, or 2 stretches may be identical and the remaining stretch may be different.

In preferred embodiments, to reduce the propensity of the molecules containing the above-discussed amino acid stretch or stretches to self-associate or self-aggregate even before being exposed to their target bacterial ESBL protein (e.g., to precipitate upon production or during storage), the amino acid stretch or stretches may be enclosed or gated by amino acids that can reduce or prevent such self-association (also termed “gatekeeper amino acids” or “gatekeepers”). Accordingly, in certain embodiments, the amino acid stretch or stretches within the molecule are each independently flanked, in particular directly or immediately flanked, on each end independently, by one or more amino acids, in particular contiguous amino acids, that display low beta-sheet forming potential or a propensity to disrupt beta-sheets. Typically, such flanking regions may each independently comprise 1 to 10, preferably 1 to 8, more preferably 1 to 6, or even more preferably 1 to 4, such as exactly 1, exactly 2, exactly 3 or exactly 4 amino acids, particularly contiguous amino acids, that have low beta-sheet forming potential or propensity to disrupt beta-sheets.

In certain preferred embodiments, an amino acid having low beta-sheet forming potential or propensity to disrupt beta-sheets may be a charged amino acid, such as a positively charged (basic, such as overall +1 or +2 charge) amino acid or a negatively charged (acidic, such as overall −1 or −2 charge) amino acid, such as an amino acid containing an amino group (—NH₃⁺ when protonated) or a carboxyl group (—COO⁻ when dissociated) in its R moiety. In certain other embodiments, an amino acid having low beta-sheet forming potential or propensity to disrupt beta-sheets may be an amino acid typified by high conformational rigidity, for example due to the inclusion of its peptide bond-forming amino group in a heterocycle, such as in pyrrolidine.

Hence, in certain preferred embodiments, an amino acid having low beta-sheet forming potential or propensity to disrupt beta-sheets may be R, K, E, D or P including D- and L-stereoisomers thereof, or analogues thereof. Accordingly, in certain embodiments, the amino acid stretch or stretches within the molecule are each independently flanked, on each end independently, by one or more amino acids, preferably by 1 to 4 contiguous amino acids, selected from the group consisting of R, K, E, D, and P, D- and L-stereoisomers thereof, and analogues thereof, and combinations thereof.

By means of an example and without limitation, an arginine analogue, in particular an arginine analogue that carries a positive charge or can be protonated to carry a positive charge, may be selected from the list consisting of 2-amino-3-ureido-propionic acid, norarginine, 2-amino-3-guanidino-propionic acid, glyoxal-hydroimidazolone, methylglyoxal-hydroimidazolone, N′-nitro-arginine, homoarginine, omega-methyl-arginine, N-alpha-methyl-arginine, N,N′-diethyl-homoarginine, canavanine, and beta-homoarginine, including their D- and L-stereoisomers, provided their structure allows such stereoisomeric forms. By means of an example and without limitation, a lysine analogue, in particular a lysine analogue that carries a positive charge or can be protonated to carry a positive charge, may be selected from the list consisting of N-epsilon-formyl-lysine, N-epsilon-methyl-lysine, N-epsilon-i-propyl-lysine, N-epsilon-dimethyl-lysine, N-epsilon-trimethylamonium-lysine, N-epsilon-nicotinyl-lysine, ornithine, N-delta-methyl-ornithine, N-delta-N-delta-dimethyl-ornithine, N-delta-i-propyl-ornithine, c-alpha-methyl-ornithine, beta, beta-dimethyl-ornithine, N-delta-methyl-N-delta-butyl-ornithine, N-delta-methyl-N-delta-phenyl-ornithine, c-alpha-methyl-lysine, beta, beta-dimethyl-lysine, N-alpha-methyl-lysine, homolysine, and beta-homolysine, including their D- and L-stereoisomers, provided their structure allows such stereoisomeric forms. By means of an example and without limitation, a glutamic or aspartic acid analogue, in particular a glutamic or aspartic acid analogue that carries a negative charge or can dissociate to carry a negative charge, may be selected from the list consisting of 2-amino-adipic acid (homoglutamic acid), 2-amino-heptanedioic acid (2-aminopimelic acid), 2-amino-octanedioic acid (aminosuberic acid), and 2-amino-4-carboxy-pentanedioic acid (4-carboxyglutamic acid), including their D- and L-stereoisomers, provided their structure allows such stereoisomeric forms. By means of an example and without limitation, a proline analogue may be selected from the list consisting of 3-methylproline, 3,4-dehydro-proline, 2-[(2S)-2-(hydrazinecarbonyl)pyrrolidin-1-yl]-2-oxoacetic acid, beta-homoproline, alpha-methyl-proline, hydroxyproline, 4-oxo-proline, beta, beta-dimethyl-proline, 5,5-dimethyl-proline, 4-cyclohexyl-proline, 4-phenyl-proline, 3-phenyl-proline, and 4-aminoproline, including their D- and L-stereoisomers, provided their structure allows such stereoisomeric forms.

By means an illustration and without limitation, examples of such gatekeeper sequences or regions that can flank the molecule stretches may be, each independently, R, K, E, D, P, RR, KK, EE, DD, PP, RK, KR, ED, DE, RRR, KKK, DDD, EEE, PPP, RRK, RKK, KKR, KRR, RKR, KRK, DDE, DEE, EED, EDD, EDE, or DED, etc., wherein any arginine, lysine, glutamate, aspartate or proline may be L- or D-isomer, and optionally wherein any arginine, lysine, glutamate, aspartate or proline may be substituted by its analogue as discussed elsewhere in this specification.

As discussed earlier, the molecules can comprise at least one portion that can assume or mimic a beta-strand conformation capable of interacting with the beta-strand contributed by the bacterial ESBL protein so as to give rise to an intermolecular beta-sheet formed by said interacting beta-strands, while in certain embodiments, such portion may preferably be an amino acid stretch (‘molecule stretch’) which participates in the intermolecular beta-sheet. In certain other embodiments, the portion may be a peptidomimetic of such a molecule stretch. The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

In certain embodiments, where the molecule comprises two or more bacterial ESBL-interacting molecule stretches as discussed herein, each optionally and preferably flanked by gatekeeper regions, these molecule stretches are connected, in particular covalently connected, directly or preferably through a linker (also known as spacer). The incorporation of such linkers or spacers may endow the individual molecule stretches with more conformational freedom and less steric hindrance to interact with the bacterial ESBL protein. Optionally, in addition to being interposed between the molecule stretches, linkers may also be added outside of the first and/or outside of the last molecule stretch of the molecule. This applies mutatis mutandis for molecules only including one molecule stretch, optionally and preferably flanked by gatekeeper regions, wherein linkers may be coupled to one or both ends of the single molecule stretch.

The nature and structure of such linkers is not particularly limited. The linker may be a rigid linker or a flexible linker. In particular embodiments, the linker is a covalent linker, achieving a covalent bond. The terms “covalent” or “covalent bond” refer to a chemical bond that involves the sharing of one or more electron pairs between two atoms. A linker may be, for example, a (poly)peptide or non-peptide linker, such as a non-peptide polymer, such as a non-biological polymer. Preferably, any linkages may be hydrolytically stable linkages, i.e., substantially stable in water at useful pH values, including in particular under physiological conditions, for an extended period of time, e.g., for days.

In certain embodiments, each linker may be independently selected from a stretch of between 1 and 20 identical or non-identical units, wherein a unit is an amino acid, a monosaccharide, a nucleotide or a monomer. Non-identical units can be non-identical units of the same nature (e.g. different amino acids, or some copolymers). They can also be non-identical units of a different nature, e.g. a linker with amino acid and nucleotide units, or a heteropolymer (copolymer) comprising two or more different monomeric species. According to specific embodiments, each linker may be independently composed of 1 to 5 units of the same nature. According to particular embodiments, all linkers present in the molecule may be of the same nature, or may be identical.

In particular embodiments, any one linker may be a peptide or polypeptide linker of one or more amino acids. In certain embodiments, all linkers in the molecule may be peptide or polypeptide linkers. More particularly, the peptide linker may be 1 to 10 amino acids long, such as more preferably 1 to 5 amino acids long. For example, the linker may be exactly 1, 2, 3, 4 or 5 amino acids long, such as preferably exactly 1, 2, 3 or 4 amino acids long. The nature of amino acids constituting the linker is not of particular relevance so long as the biological activity of the molecule stretches linked thereby is not substantially impaired. Preferred linkers are essentially non-immunogenic and/or not prone to proteolytic cleavage. In certain embodiments, the linker may contain a predicted secondary structure such as an alpha-helical structure. However, linkers predicted to assume flexible, random coil structures are preferred. Linkers having tendency to form beta-strands may be less preferred or may need to be avoided. Cysteine residues may be less preferred or may need to be avoided due to their capacity to form intermolecular disulphide bridges. Basic or acidic amino acid residues, such as arginine, lysine, histidine, aspartic acid and glutamic acid may be less preferred or may need to be avoided due to their capacity for unintended electrostatic interactions. In certain preferred embodiments, the peptide linker may comprise, consist essentially of or consist of amino acids selected from the group consisting of glycine, serine, alanine, threonine, proline, and combinations thereof, including D-isomers and analogues thereof. In even more preferred embodiments, the peptide linker may comprise, consist essentially of or consist of amino acids selected from the group consisting of glycine, serine, and combinations thereof, including D-isomers and analogues thereof. In certain embodiments, the peptide linker may consist of only glycine and serine residues. In certain embodiments, the peptide linker may consist of only glycine residues or analogues thereof, preferably of only glycine residues. In certain embodiments, the peptide linker may consist of only serine residues or D-isomers or analogues thereof, preferably of only serine residues. Such linkers provide for particularly good flexibility. In certain embodiments, the linker may consist essentially of or consist of glycine and serine residues. In certain embodiments, the glycine and serine residues may be present at a ratio between 4:1 and 1:4 (by number), such as about 3:1, about 2:1, about 1:1, about 1:2 or about 1:3 glycine:serine. Preferably, glycine may be more abundant than serine, e.g., a ratio between 4:1 and 1.5:1 glycine:serine, such as about 3:1 or about 2:1 glycine:serine (by number). In certain embodiments, the N-terminal and C-terminal residues of the linker are both a serine residue; or the N-terminal and C-terminal residues of the linker are both glycine residues; or the N-terminal residue is a serine residue and the C-terminal residue is a glycine residue; or the N-terminal residue is a glycine residue and the C-terminal residue is a serine residue. In certain embodiments, the peptide linker may consist of only proline residues or D-isomers or analogues thereof, preferably of only proline residues. By means of examples and without limitation, peptide linkers as intended herein may be e.g. P, PP, PPP, GS, SG, SGG, SSG, GSS, GGS or GSGS etc.

In certain embodiments, the linker may be a non-peptide linker. In preferred embodiments, the non-peptide linker may comprise, consist essentially of or consist of a non-peptide polymer. The term “non-peptide polymer” as used herein refers to a biocompatible polymer including two or more repeating units linked to each other by a covalent bond excluding the peptide bond. For example, the non-peptide polymer may be 2 to 200 units long or 2 to 100 units long or 2 to 50 units long or 2 to 45 units long or 2 to 40 units long or 2 to 35 units long or 2 to 30 units long or 5 to 25 units long or 5 to 20 units long or 5 to 15 units long. The non-peptide polymer may be selected from the group consisting of polyethylene glycol, polypropylene glycol, copolymers of ethylene glycol and propylene glycol, polyoxyethylated polyols, polyvinyl alcohol, polysaccharides, dextran, polyvinyl ethyl ether, biodegradable polymers such as PLA (poly(lactic acid) and PLGA (polylactic-glycolic acid), lipid polymers, chitins, hyaluronic acid, and combinations thereof. Particularly preferred is poly(ethylene glycol) (PEG). Another particularly envisaged chemical linker is Ttds (4,7,10-trioxatridecan-13-succinamic acid). The molecular weight of the non-peptide polymer preferably may range from 1 to 100 kDa, and preferably 1 to 20 kDa. The non-peptide polymer may be one polymer or a combination of different types of polymers. The non-peptide polymer has reactive groups capable of binding to the elements which are to be coupled by the linker. Preferably, the non-peptide polymer has a reactive group at each end. Preferably, the reactive group is selected from the group consisting of a reactive aldehyde group, a propione aldehyde group, a butyl aldehyde group, a maleimide group and a succinimide derivative. The succinimide derivative may be succinimidyl propionate, hydroxy succinimidyl, succinimidyl carboxymethyl or succinimidyl carbonate. The reactive groups at both ends of the non-peptide polymer may be the same or different. In certain embodiments, the non-peptide polymer has a reactive aldehyde group at both ends. For example, the non-peptide polymer may possess a maleimide group at one end and, at the other end, an aldehyde group, a propionic aldehyde group or a butyl aldehyde group. When a polyethylene glycol (PEG) having a reactive hydroxy group at both ends thereof is used as the non-peptide polymer, the hydroxy group may be activated to various reactive groups by known chemical reactions, or a PEG having a commercially-available modified reactive group may be used so as to prepare the protein conjugate.

In certain particularly preferred embodiments, the operative part of the molecule, i.e., the part responsible for the effects on the bacterial ESBL protein, may be a peptide. Put differently, in such embodiments, the molecule stretch or stretches that form beta-strands interacting with the APR of the bacterial ESBL protein, the optional and preferred flanking gatekeeper regions, the linkers optionally and preferably interposed between the molecule stretches, and the linkers optionally but less preferably added outside of the outermost molecule stretches, are all composed of amino acids (which may include D- and L-stereoisomers and amino acid analogues) covalently linked by peptide bonds. Preferably, the total length of such peptide operative part of the molecule does not exceed 50 amino acids, such as does not exceed 45, 40, 35, 30, 25 or even 20 amino acids. Such peptide operative part of the molecule may be coupled to one or more other moieties, which themselves may but need not be amino acids, peptides, or polypeptides, and which may serve other functions, such as allowing to detect the molecule, increasing the half-life of the molecule when administered to subjects, increasing the solubility of the molecule, increasing the cellular uptake of the molecule, etc., as discussed elsewhere in this specification. In certain particularly preferred embodiments, the molecule is a peptide. Preferably, the total length of such peptide does not exceed 50 amino acids, such as does not exceed 45, 40, 35, 30, 25 or even 20 amino acids. Where the molecule comprises, consists essentially of or consists of, e.g., is, a peptide the N-terminus of said molecule can be modified, such as for example by acetylation, and/or the C-terminus of said molecule can be modified, such as for example by amidation.

In view of the foregoing discussion, in certain embodiments, the molecule as taught herein may be conveniently represented as comprising, consisting essentially of or consisting of the structure:
a) NGK1-P1-CGK1,
b) NGK1-P1-CGK1-Z1-NGK2-P2-CGK2,
c) NGK1-P1-CGK1-Z1-NGK2-P2-CGK2-Z2-NGK3-P3-CGK3,
   wherein:
P1 to P3 each independently denote the amino acid stretch (‘molecule stretch’) as taught above,
NGK1 to NGK4 and CGK1 to CGK4 each independently denote the gatekeeper region as taught above, and
Z1 to Z3 each independently denote the linker as taught above.

Hence, structure a) refers to a molecule only containing one molecule stretch as taught herein, while structures b) and c) refer to molecules containing a two or three molecule stretch as taught herein, respectively.

In certain embodiments, as explained above, NGK1 to NGK4 and CGK1 to CGK4 may each independently denote 1 to 4 contiguous amino acids that display low beta-sheet forming potential or a propensity to disrupt beta-sheets, preferably 1 to 4 contiguous amino acids selected from the group consisting of R, K, D, E and P, D-isomers and/or analogues thereof, and combinations thereof. In certain particularly preferred embodiments, NGK1 to NGK4 and CGK1 to CGK4 may each independently denote 1 to 2 contiguous amino acids selected from the group consisting of R, K, and D, D-isomers and/or analogues thereof, and combinations thereof, such as NGK1 to NGK4 and CGK1 to CGK4 may be each independently K, R, D or KK.

In some instances in the peptides, the N-terminal amino acid may be modified such as acetylated and/or the C-terminal amino acid may be modified such as amidated. In such peptides, D-amino acid(s) and or amino acid analogue(s) can be incorporated as long as their incorporation is compatible with the formation of the intermolecular beta-sheet as taught herein.

In certain embodiments, the molecule as taught herein may comprise one or more further moieties, groups, components or parts, which may serve other functions or perform other roles and activities. Such functions, roles or activities may be useful or desired for example in connection with the production, synthesis, isolation, purification or formulation of the molecule, or in connection with its in experimental or therapeutic uses. Conveniently, the operative part of the molecule, i.e., the part responsible for the effects on the bacterial ESBL protein, may be connected to one or more such further moieties, groups, components or parts, preferably covalently connected, bound, linked or fused, directly or through a linker. Where such further moiety, group, component or part is a peptide, polypeptide or protein, the connection to the operative part of the molecule may preferably involve a peptide bond, direct one or through a peptide linker.

For all such added moieties, the nature of the fusion or linker is not vital to the invention, as long as the moiety and the molecule can exert their specific function. According to particular embodiments, the moieties which are fused to the molecules can be cleaved off, e.g. by using a linker moiety that has a protease recognition site. This way, the function of the moiety and the molecule can be separated, which may be particularly interesting for larger moieties, or for embodiments where the moiety is no longer necessary after a specific point in time, e.g., a tag that is cleaved off after a separation step using the tag.

In certain preferred embodiments, the molecule may comprise a detectable label, a moiety that allows for isolation of the molecule, a moiety increasing the stability of the molecule, a moiety increasing the solubility of the molecule, a moiety increasing the cellular uptake of the molecule, a moiety effecting targeting of the molecule to cells, or a combination of any two or more thereof. It shall be appreciated that a single moiety can carry out two or more functions or activities.

Hence, in certain embodiments the molecule may comprise a detectable label. The term “label” refers to any atom, molecule, moiety or biomolecule that may be used to provide a detectable and preferably quantifiable read-out or property, and that may be attached to or made part of an entity of interest, such as molecules as taught herein, such as peptides as taught herein. Labels may be suitably detectable by for example mass spectrometric, spectroscopic, optical, colourimetric, magnetic, photochemical, biochemical, immunochemical or chemical means. Labels include without limitation dyes; radiolabels such as isotopes of hydrogen, carbon, nitrogen, oxygen, phosphorous, sulphur, fluorine, chlorine, or iodine, such as ²H, ³H, ¹³C, ¹¹C, ¹⁴C, ¹⁵N, ¹⁸O, ¹⁷O, ³¹P, ³²P, ³³P, ³⁵S, ¹⁸F, ³⁶Cl, ¹²⁵I, or ¹³¹I respectively; electron-dense reagents; enzymes (e.g., horse-radish peroxidase or alkaline phosphatase as commonly used in immunoassays); binding moieties such as biotin-streptavidin; haptens such as digoxigenin; luminogenic, phosphorescent or fluorogenic moieties; mass tags; fluorescent dyes (e.g., fluorophores such as fluorescein, carboxyfluorescein (FAM), tetrachloro-fluorescein, TAMRA, ROX, Cy3, Cy3.5, Cy5, Cy5.5, Texas Red, etc.) alone or in combination with moieties that may suppress or shift emission spectra by fluorescence resonance energy transfer (FRET); and fluorescent proteins (e.g., GFP, RFP). Certain isotopically labelled molecules such as peptides as taught herein, for example those into which radioactive isotopes such as ³H and ¹⁴C are incorporated, are useful in drug and/or substrate tissue distribution assays. ³H and ¹⁴C isotopes are particularly preferred for their ease of preparation and detectability. Further, substitution with heavier isotopes such as 2H may afford certain therapeutic advantages resulting from greater metabolic stability, for example increased in vivo half-life or reduced dosage requirements and, hence, may be preferred in some circumstances. Isotopically labelled molecules such as peptides may generally be prepared by carrying production or synthesis methods in which a readily available isotopically labelled reagent is substituted for a non-isotopically labelled reagent. In some embodiments, the molecule may be provided with a tag that permits detection with another agent (e.g., with a probe binding partner). Such tags may be, for example, biotin, streptavidin, his-tag, myc tag, FLAG tag, maltose, maltose binding protein or any other kind of tag known in the art that has a binding partner. Example of associations which may be utilised in the probe:binding partner arrangement may be any, and includes, for example biotin:streptavidin, his-tag:metal ion (e.g., Ni²⁺), maltose:maltose binding protein, etc.

In further embodiments, the molecule may comprise a moiety that allows for the isolation (separation, purification) of the molecule. Typically, such moieties operate in conjunction with affinity purification methods, in which the ability to isolate a particular component of interest from other components is conferred by specific binding between a separable binding agent, such as an immunological binding agent (antibody), and the component of interest. Such affinity purification methods include without limitation affinity chromatography and magnetic particle separation. Such moieties are well-known in the art and non-limiting examples include biotin (isolatable using an affinity purification method utilising streptavidin), his-tag (isolatable using an affinity purification method utilising metal ion, e.g., Ni²⁺), maltose (isolatable using an affinity purification method utilising maltose binding protein), glutathione S-transferase (GST) (isolatable using an affinity purification method utilising glutathione), or myc or FLAG tag (isolatable using an affinity purification method utilising anti-myc or anti-FLAG antibody, respectively).

In further embodiments, the molecule may comprise a moiety that increases the solubility of the molecule. While the solubility of the molecules can be ensured and controlled by the inclusion of gatekeeper portions flanking the molecule stretch or stretches as discussed above, whereby this may in principle be sufficient to prevent premature aggregation of the molecules and keep them in solution, the further addition of a moiety that increases solubility, i.e., prevents aggregation, may provide easier handling of the molecules, and particularly improve their stability and shelf-life. Many of the labels and isolation tags discussed above will also increase the solubility of the molecule. Further, a well-known example of such solubilising moiety is PEG (polyethylene glycol). This moiety is particularly envisaged, as it can be used as linker as well as solubilising moiety. Other examples include peptides and proteins or protein domains, or even whole proteins, e.g. GFP. In this regard, it should be noted that, like PEG, one moiety can have different functions or effects. For instance, a FLAG tag is a peptide moiety that can be used as a label, but due to its charge density, it will also enhance solubilisation. PEGylation has already often been demonstrated to increase solubility of biopharmaceuticals (e.g., Veronese and Mero, BioDrugs. 2008; 22(5):315-29). Adding a peptide, polypeptide, protein or protein domain tag to a molecule of interest has been extensively described in the art. Examples include, but are not limited to, peptides derived from synuclein (e.g., Park et al., Protein Eng. Des. Sel. 2004; 17:251-260), SET (solubility enhancing tag, Zhang et al., Protein Expr Purif 2004; 36:207-216), thioredoxin (TRX), Glutathione-S-transferase (GST), Maltose-binding protein (MBP), N-Utilization substance (NusA), small ubiquitin-like modifier (SUMO), ubiquitin (Ub), disulfide bond C (DsbC), Seventeen kilodalton protein (Skp), Phage T7 protein kinase fragment (T7PK), Protein G B1 domain, Protein A IgG ZZ repeat domain, and bacterial immunoglobulin binding domains (Hutt et al., J Biol Chem.; 287(7):4462-9, 2012). The nature of the tag will depend on the application, as can be determined by the skilled person. For instance, for transgenic expression of the molecules described herein, it might be envisaged to fuse the molecules to a larger domain to prevent premature degradation by the cellular machinery. Other applications may envisage fusion to a smaller solubilisation tag (e.g., less than 30 amino acids, or less than 20 amino acids, or even less than 10 amino acids) in order not to alter the properties of the molecules too much.

In further embodiments, the molecule may comprise a moiety increasing the stability of the molecule, e.g., the shelf-life of the molecule, and/or the half-life of the molecule, which may involve increasing the stability of the molecule and/or reducing the clearance of the molecule when administered. Such moieties may modulate pharmacokinetic and pharmacodynamic properties of the molecule. Many of the labels, isolation tags and solubilisation tags discussed above will also increase the shelf-life or in vivo half-life of the molecules. For instance, it is known that fusion with albumin (e.g., human serum albumin), albumin-binding domain or a synthetic albumin-binding peptide improves pharmacokinetics and pharmacodynamics of different therapeutic proteins (Langenheim and Chen, Endocrinol.; 203(3):375-87, 2009). Another moiety that is often used is a fragment crystallizable region (Fc) of an antibody. Strohl (BioDrugs. 2015, vol. 29, 215-39) reviews fusion protein-based strategies for half-life extension of biologics, including without limitation fusion to human IgG Fc domain, fusion to HSA, fusion to human transferrin, fusion to artificial gelatin-like protein (GLP), etc. In particular embodiments, the molecules are not fused to an agarose bead, a latex bead, a cellulose bead, a magnetic bead, a silica bead, a polyacrylamide bead, a microsphere, a glass bead or any solid support (e.g. polystyrene, plastic, nitrocellulose membrane, glass), or the NusA protein. However, these fusions are possible, and in specific embodiments, they are also envisaged.

As mentioned, in particular embodiments, the operative part of the molecule may comprise, consist essentially of or consist of a peptide, preferably the operative part of the molecule may be a peptide. Moreover, in many embodiments, for example, where the operative part of the molecule is not connected or fused to other auxiliary moieties or where such additional moiety or moieties are themselves peptides, the entire molecule may be a peptide. Accordingly, standards tools and methods of chemical peptide synthesis, or of recombinant peptide or polypeptide production can be applied to the preparation of the present molecules. Recombinant protein production can also be applied to preparing molecules in which additional moiety or moieties which are themselves proteinaceous are included in the molecules and fused to the operative part of the molecule by peptide bonds.

Given that such techniques have become generally routine, in the interest of brevity, recombinant production of the present molecules may employ an expression cassette or expression vector comprising a nucleic acid encoding the molecule as taught herein and a promoter operably linked to the nucleic acid, wherein the expression cassette or expression vector is configured to effect expression of the molecule in a suitable host cell, such as a bacterial cell, a fungal cell, including yeast cells, an animal cell, or a mammalian cell, including human cells and non-human mammalian cells.

Any molecules, such as proteins, polypeptides or peptides as prepared herein can be suitably purified. The term “purified” with reference to molecules, peptides, polypeptides or proteins does not require absolute purity. Instead, it denotes that such molecules, peptides, polypeptides or proteins are in a discrete environment in which their abundance (conveniently expressed in terms of mass or weight or concentration) relative to other components is greater than in the starting composition or sample, e.g., in the production sample, such as in a lysate or supernatant of a recombinant host cells producing the molecule, peptide, polypeptide or protein. A discrete environment denotes a single medium, such as for example a single solution, gel, precipitate, lyophilisate, etc. Purified molecules, proteins, polypeptides or peptides may be obtained by known methods including, for example, chemical synthesis, chromatography, preparative electrophoresis, centrifugation, precipitation, affinity purification, etc. Purified molecules, peptides, polypeptides or proteins may preferably constitute by weight ≥10%, more preferably ≥50%, such as ≥60%, yet more preferably ≥70%, such as ≥80%, and still more preferably ≥90%, such as ≥95%, ≥96%, ≥97%, ≥98%, ≥99% or even 100%, of the non-solvent content of the discrete environment. For example, purified peptides, polypeptides or proteins may preferably constitute by weight ≥10%, more preferably ≥50%, such as ≥60%, yet more preferably ≥70%, such as ≥80%, and still more preferably ≥90%, such as ≥95%, ≥96%, ≥97%, ≥98%, ≥99% or even 100%, of the protein content of the discrete environment. Protein content may be determined, e.g., by the Lowry method (Lowry et al. 1951. J Biol Chem 193: 265), optionally as described by Hartree 1972 (Anal Biochem 48: 422-427). Purity of peptides, polypeptides, or proteins may be determined by HPLC, or SDS-PAGE under reducing or non-reducing conditions using Coomassie blue or, preferably, silver stain.

Any molecules, such as proteins, polypeptides or peptides as prepared herein can be suitably kept in solution in deionised water, or in deionised water with DMSO, e.g., 50% v/v DMSO in deionised water, or in an aqueous solution, or in a suitable buffer, such as in a buffer having physiological pH, or at pH between 5 and 9, more particular pH between 6 and 8, such as in neutral buffered saline, phosphate buffered saline, Tris-HCl, acetate or phosphate buffers, or in a strong chaotropic agent such as 6M urea, at concentrations of the molecules convenient for downstream use, such as without limitation between about 1 mM and about 500 mM, or between about 1 mM and about 250 mM, or between about 1 mM and about 100 mM, or between about 5 mM and about 50 mM, or between about 5 mM and about 20 mM. Alternatively, any molecules, such as proteins, polypeptides or peptides as prepared herein may be lyophilised as is generally known in the art. Storage may typically be at or below room temperature (at or below 25° C. ), in certain embodiments at temperatures above 0° C. (non-cryogenic storage), such as at a temperature above 0° C. and not exceeding 25° C., or in certain embodiments cryopreservation may be preferred, at temperatures of 0° C. or lower, typically −5° C. or lower, more typically −10° C. or lower, such as −20° C. or lower, −25° C. or lower, −30° C. or lower, or even at −70° C. or lower or −80° C. or lower, or in liquid nitrogen.

The molecules as taught herein are useful for therapy. Hence, an aspect provides any molecule as taught herein for use in medicine, or in other words, any molecule as taught herein for use in therapy. As discussed below, the molecules as taught herein can be formulated into pharmaceutical compositions. Therefore, any reference to the use of the molecules in therapy (or any variation of such language) also subsumes the use of pharmaceutical compositions comprising the molecules in therapy.

In particular, the molecules are intended for therapy of afflictions in mammalians, such as humans in which bacterial infections occur.

Reference to “therapy” or “treatment” broadly encompasses both curative and preventative treatments, and the terms may particularly refer to the alleviation or measurable lessening of one or more symptoms or measurable markers of a pathological condition such as a disease or disorder.

The terms “subject”, “individual” or “patient” are used interchangeably throughout this specification, and typically and preferably denote humans, but may also encompass reference to non-human animals, preferably warm-blooded animals, even more preferably non-human mammals. Particularly preferred are human subjects including both genders and all age categories thereof. In other embodiments, the subject is an experimental animal or animal substitute as a disease model. The term does not denote a particular age or sex. Thus, adult and new-born subjects, as well as foetuses, whether male or female, are intended to be covered. The term subject is further intended to include transgenic non-human species.

The term “subject in need of treatment” or similar as used herein refers to subjects diagnosed with or having a disease as recited herein and/or those in whom said disease is to be prevented.

The term “therapeutically effective amount” generally denotes an amount sufficient to elicit the pharmacological effect or medicinal response in a subject that is being sought by a medical practitioner such as a medical doctor, clinician, surgeon, veterinarian, or researcher, which may include inter alia alleviation of the symptoms of the disease being treated, in either a single or multiple doses. Appropriate therapeutically effective doses of the present molecules may be determined by a qualified physician with due regard to the nature and severity of the disease, and the age and condition of the patient. The effective amount of the molecules described herein to be administered can depend on many different factors and can be determined by one of ordinary skill in the art through routine experimentation. Several non-limiting factors that might be considered include biological activity of the active ingredient, nature of the active ingredient, characteristics of the subject to be treated, etc. The term “to administer” generally means to dispense or to apply, and typically includes both in vivo administration and ex vivo administration to a tissue, preferably in vivo administration. Generally, compositions may be administered systemically or locally.

In certain embodiments, any molecule as taught herein may be administered as the sole pharmaceutical agent (active pharmaceutical ingredient) or in combination with one or more other pharmaceutical agents where the combination causes no unacceptable adverse effects. By means of an example, two or more molecules as taught herein may be co-administered. By means of another example, one or more molecules as taught herein may be co-administered with a pharmaceutical agent that is not a molecule as envisaged herein.

### Combinations of the Non-Natural Molecules of the Invention and Beta-Lactam Antibiotics

Beta-lactam antibiotics are antibiotics that contain a beta-lactam ring in their molecular structure. This includes penicillin derivatives (penams), cephalosporins (cephems), monobactams, clavams, carbapenems, oxacephems and carbacephems.

Penams are classified as beta-lactams fused to saturated five-membered rings and the rings are thiazolidine rings, examples are benzathine, benzylpenicillin (penicillin G), benzathine penicillin G, benzathine penicillin V, phenoxtmethylpenicillin (penicillin V), procaine penicillin and pheneticillin, cloxacillin, dicloxacillin, flucloxacillin, methicillin, nafcillin, oxacillin, temocillin, amoxicillin, ampicillin, mecillinam, piperacillin, carbenicillin, ticarcillin, carbenicillin, ticarcillin, azlocillin, mezlocillin and piperacillin.

Cephems are classified as beta-lactams fused to unsaturated six-membered rings and the rings are 3,6-dihydro-2H-1,3-thiazine rings, examples are cefazolin, cephalexin, cephalosporin C, cephalothin, cefapirin, cefaclor, cefamandole, cefuroxime, cefotetan, cefoxitin, cefixime, cefotaxime, cefpodoxime, ceftazidime ceftriaxone, cefdinir, cefepime, cefpirome and ceftaroline.

Penems are classified as beta-lactams fused to unsaturated five-membered rings and the rings are 2,3-dihydrothizazole rings for the penems and 2,3-dihydro-1H-pyrrole rings for the carbapenems, examples are biapenem, doripenem, ertapenem, faropenem, imipenem, meropenem, panipenem, razupenem, tebipenem and thienamycin.

Monobactams are classified as beta-lactams not fused to any other ring structure, examples are aztreonam, tigemonam, nocardicin A and tabtoxinine beta-lactam.

Clavams are classified as beta-lactams fused to saturated five-membered rings and the rings are oxapenams, examples are lavulanic acid, clavamycin A and valclavam) and carbapenems [olivanic acids, thienamycin, imipenem (a derivative of thienamycin, N-formimidoylthienamycin), meropenem and 1-carbapen-2-em-3-carboxylic acid].

Carbacephems are classified as beta-lactams fused to unsaturated six-membered rings and the rings are 1, 2, 3, 4-tetrahydropyridine rings, examples are penam (Sulbactam), Tazobactam, Clavam (Clavulanic acid), relebactam, avibactam and vaborbactam.

Oxacephems are beta-lactams fused to unsaturated six-membered rings and the rings are 3, 6-dihydro-2H-1,3-oxazine rings, examples are Cefaclor, Cefotetan, Cephamycin (Cefoxitin), Cefprozil, Cefuroxime, Cefuroxime axetil, Cefamandole and Cefminox.

### Combinations of the Non-Natural Molecules of the Invention With Beta-Lactam Antibiotics and Beta-Lactamase Inhibitors

Examples of beta-lactamase inhibitors are clavulanic acid, tazobactam, sulfabactam and avibactam.

For example, the reference to the molecule as intended herein may encompass a given therapeutically useful compound as well as any pharmaceutically acceptable forms of such compound, such as any addition salts, hydrates or solvates of the compound. The term “pharmaceutically acceptable” as used herein inter alia in connection with salts, hydrates, solvates and excipients, is consistent with the art and means compatible with the other ingredients of a pharmaceutical composition and not deleterious to the recipient thereof. Pharmaceutically acceptable acid and base addition salts are meant to comprise the therapeutically active non-toxic acid and base addition salt forms which the compound is able to form. The pharmaceutically acceptable acid addition salts can conveniently be obtained by treating the base form of a compound with an appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form. A compound containing an acidic proton may also be converted into its non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, aluminum salts, zinc salts, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form. The term solvate comprises the hydrates and solvent addition forms which the compound is able to form, as well as the salts thereof. Examples of such forms are, e.g., hydrates, alcoholates and the like.

For example, the molecule may be a part of a composition. The term “composition” generally refers to a thing composed of two or more components, and more specifically particularly denotes a mixture or a blend of two or more materials, such as elements, molecules, substances, biological molecules, or microbiological materials, as well as reaction products and decomposition products formed from the materials of the composition. By means of an example, a composition may comprise any molecule as taught herein in combination with one or more other substances. For example, a composition may be obtained by combining, such as admixing, the molecule as taught herein with said one or more other substances. In certain embodiments, the present compositions may be configured as pharmaceutical compositions. Pharmaceutical compositions typically comprise one or more pharmacologically active ingredients (chemically and/or biologically active materials having one or more pharmacological effects) and one or more pharmaceutically acceptable carriers. Compositions as typically used herein may be liquid, semisolid or solid, and may include solutions or dispersions.

Hence, a further aspect provides a pharmaceutical composition comprising any molecule as taught herein. The terms “pharmaceutical composition” and “pharmaceutical formulation” may be used interchangeably. The pharmaceutical compositions as taught herein may comprise in addition to the one or more actives, one or more pharmaceutically or acceptable carriers. Suitable pharmaceutical excipients depend on the dosage form and identities of the active ingredients and can be selected by the skilled person (e.g., by reference to the Handbook of Pharmaceutical Excipients 7^{th} Edition 2012, eds. Rowe et al.).

As used herein, the terms “carrier” or “excipient” are used interchangeably and broadly include any and all solvents, diluents, buffers (such as, e.g., neutral buffered saline, phosphate buffered saline, or optionally Tris-HCl, acetate or phosphate buffers), solubilisers (such as, e.g., Tween® 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives (such as, e.g., Thimerosal™, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of such media and agents for the formulation of pharmaceutical and cosmetic compositions is well known in the art. Acceptable diluents, carriers and excipients typically do not adversely affect a recipient's homeostasis (e.g., electrolyte balance). The use of such media and agents for pharmaceutical active substances is well known in the art. Such materials should be non-toxic and should not interfere with the activity of the actives. Acceptable carriers may include biocompatible, inert or bioabsorbable salts, buffering agents, oligo- or polysaccharides, polymers, viscosity-improving agents, preservatives and the like. One exemplary carrier is physiologic saline (0.15 M NaCl, pH 7.0 to 7.4). Another exemplary carrier is 50 mM sodium phosphate, 100 mM sodium chloride.

The precise nature of the carrier or other material will depend on the route of administration. For example, the pharmaceutical composition may be in the form of a parenterally acceptable aqueous solution, which is pyrogen-free and has suitable pH, isotonicity and stability.

The pharmaceutical formulations may comprise pharmaceutically acceptable auxiliary substances as required to approximate physiological conditions, such as pH adjusting and buffering agents, preservatives, complexing agents, tonicity adjusting agents, wetting agents and the like, for example, sodium acetate, sodium lactate, sodium phosphate, sodium hydroxide, hydrogen chloride, benzyl alcohol, parabens, EDTA, sodium oleate, sodium chloride, potassium chloride, calcium chloride, sorbitan monolaurate, triethanolamine oleate, etc. Preferably, the pH value of the pharmaceutical formulation is in the physiological pH range, such as particularly the pH of the formulation is between about 5 and about 9.5, more preferably between about 6 and about 8.5, even more preferably between about 7 and about 7.5.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art. Liposomes are artificial membrane vesicles which are useful as delivery vehicles in vitro and in vivo. These formulations may have net cationic, anionic or neutral charge characteristics and are useful characteristics with in vitro, in vivo and ex vivo delivery methods. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Pharmaceutical compositions as intended herein may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous (I.V.), intramuscular, intraperitoneal or intrasternal injection or infusion), transdermal or transmucosal (such as, e.g., oral, sublingual, intranasal) administration, topical administration, rectal, vaginal or intra-tracheal instillation, and the like. In this way, the therapeutic effects attainable by the methods and compositions can be, for example, systemic, local, tissue-specific, etc., depending of the specific needs of a given application.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the agent as disclosed herein in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing the agent as disclosed herein with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

For example, for oral or nasal aerosol or inhalation administration, pharmaceutical compositions may be formulated with illustrative carriers, such as, e.g., as in solution with saline, polyethylene glycol or glycols, DPPC, methylcellulose, or in mixture with powdered dispersing agents, further employing benzyl alcohol or other suitable preservatives, absorption promoters to enhance bioavailability, fluorocarbons, and/or other solubilising or dispersing agents known in the art. Suitable pharmaceutical formulations for administration in the form of aerosols or sprays are, for example, solutions, suspensions or emulsions of the agents as taught herein or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Illustratively, delivery may be by use of a single-use delivery device, a mist nebuliser, a breath-activated powder inhaler, an aerosol metered-dose inhaler (MDI) or any other of the numerous nebuliser delivery devices available in the art. Additionally, mist tents or direct administration through endotracheal tubes may also be used.

Examples of carriers for administration via mucosal surfaces depend upon the particular route, e.g., oral, sublingual, intranasal, etc. When administered orally, illustrative examples include pharmaceutical grades of mannitol, starch, lactose, magnesium stearate, sodium saccharide, cellulose, magnesium carbonate and the like, with mannitol being preferred. When administered intranasally, illustrative examples include polyethylene glycol, phospholipids, glycols and glycolipids, sucrose, and/or methylcellulose, powder suspensions with or without bulking agents such as lactose and preservatives such as benzalkonium chloride, EDTA. In a particularly illustrative embodiment, the phospholipid 1,2 dipalmitoyl-sn-glycero-3-phosphocholine (DPPC) is used as an isotonic aqueous carrier at about 0.01-0.2% for intranasal administration of the compound of the subject invention at a concentration of about 0.1 to 3.0 mg/ml.

For example, for parenteral administration, pharmaceutical compositions may be advantageously formulated as solutions, suspensions or emulsions with suitable solvents, diluents, solubilisers or emulsifiers, etc. Suitable solvents are, without limitation, water, physiological saline solution, PBS, Ringer's solution, dextrose solution, or Hank's solution, or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The agents and pharmaceutically acceptable salts thereof of the invention can also be lyophilised and the lyophilisates obtained used, for example, for the production of injection or infusion preparations. For example, one illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

Where aqueous formulations are preferred, such may comprise one or more surfactants. For example, the composition can be in the form of a micellar dispersion comprising at least one suitable surfactant, e.g., a phospholipid surfactant. Illustrative examples of phospholipids include diacyl phosphatidyl glycerols, such as dimyristoyl phosphatidyl glycerol (DPMG), dipalmitoyl phosphatidyl glycerol (DPPG), and distearoyl phosphatidyl glycerol (DSPG), diacyl phosphatidyl cholines, such as dimyristoyl phosphatidylcholine (DPMC), dipalmitoyl phosphatidylcholine (DPPC), and distearoyl phosphatidylcholine (DSPC); diacyl phosphatidic acids, such as dimyristoyl phosphatidic acid (DPMA), dipahnitoyl phosphatidic acid (DPPA), and distearoyl phosphatidic acid (DSPA); and diacyl phosphatidyl ethanolamines such as dimyristoyl phosphatidyl ethanolamine (DPME), dipalmitoyl phosphatidyl ethanolamine (DPPE) and distearoyl phosphatidyl ethanolamine (DSPE). Typically, a surfactant:active substance molar ratio in an aqueous formulation will be from about 10:1 to about 1:10, more typically from about 5:1 to about 1:5, however any effective amount of surfactant may be used in an aqueous formulation to best suit the specific objectives of interest.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquify and/or dissolve in the rectal cavity to release the drug.

Suitable carriers for microcapsules, implants or rods are, for example, copolymers of glycolic acid and lactic acid.

One skilled in this art will recognise that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

The dosage or amount of the molecules as taught herein, optionally in combination with one or more other active compounds to be administered, depends on the individual case and is, as is customary, to be adapted to the individual circumstances to achieve an optimum effect. Thus, the unit dose and regimen depend on the nature and the severity of the disorder to be treated, and also on factors such as the species of the subject, the sex, age, body weight, general health, diet, mode and time of administration, immune status, and individual responsiveness of the human or animal to be treated, efficacy, metabolic stability and duration of action of the compounds used, on whether the therapy is acute or chronic or prophylactic, or on whether other active compounds are administered in addition to the agent of the invention. In order to optimize therapeutic efficacy, the molecule as taught herein can be first administered at different dosing regimens. Typically, levels of the molecule in a tissue can be monitored using appropriate screening assays as part of a clinical testing procedure, e.g., to determine the efficacy of a given treatment regimen. The frequency of dosing is within the skills and clinical judgement of medical practitioners (e.g., doctors, veterinarians or nurses). Typically, the administration regime is established by clinical trials which may establish optimal administration parameters. However, the practitioner may vary such administration regimes according to the one or more of the aforementioned factors, e.g., subject's age, health, weight, sex and medical status. The frequency of dosing can be varied depending on whether the treatment is prophylactic or therapeutic.

Toxicity and therapeutic efficacy of the molecules as described herein or pharmaceutical compositions comprising the same can be determined by known pharmaceutical procedures in, for example, cell cultures or experimental animals. These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Pharmaceutical compositions that exhibit high therapeutic indices are preferred. While pharmaceutical compositions that exhibit toxic side effects can be used, care should be taken to design a delivery system that targets such compounds to the site of affected tissue in order to minimize potential damage to normal cells (e.g., non-target cells) and, thereby, reduce side effects.

The data obtained from the cell culture assays and animal studies can be used in formulating a range of dosage for use in appropriate subjects. The dosage of such pharmaceutical compositions lies generally within a range of circulating concentrations that include the ED50 with little or no toxicity. The dosage may vary within this range depending upon the dosage form employed and the route of administration utilized. For a pharmaceutical composition used as described herein, the therapeutically effective dose can be estimated initially from cell culture assays. A dose can be formulated in animal models to achieve a circulating plasma concentration range that includes the IC50 (i.e., the concentration of the pharmaceutical composition which achieves a half-maximal inhibition of symptoms) as determined in cell culture. Such information can be used to more accurately determine useful doses in humans. Levels in plasma can be measured, for example, by high performance liquid chromatography.

Without limitation, depending on the type and severity of the disease, a typical dosage (e.g., a typical daily dosage or a typical intermittent dosage, e.g., a typical dosage for every two days, every three days, every four days, every five days, every six days, every week, every 1.5 weeks, every two weeks, every three weeks, every month, or other) of the molecules as taught herein may range from about 10 μg/kg to about 100 mg/kg body weight of the subject, per dose, depending on the factors mentioned above, e.g., may range from about 100 μg/kg to about 10 mg/kg body weight of the subject, per dose, or from about 200 μg/kg to about 2 mg/kg body weight of the subject, per dose, e.g., may be about 100 μg/kg, about 200 μg/kg, about 300 μg/kg, about 400 μg/kg, about 500 μg/kg, about 600 μg/kg, about 700 μg/kg, about 800 μg/kg, about 900 μg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, or about 2.0 mg/kg body weight of the subject. By means of example and without limitation, the molecules as taught herein may be administered at about 0.5 mg/kg, or at about 0.6 mg/kg, or at about 0.7 mg/kg, or at about 0.8 mg/kg, or at about 0.9 mg/kg, or at about 1.0 mg/kg, or at about 1.5 mg/kg, or at about 2.0 mg/kg, or at about 2.5 mg/kg, or at about 3.0 mg/kg, or at about 3.5 mg/kg, or at about 4.0 mg/kg.

In particular embodiments, the molecule as taught herein is administered using a sustained delivery system, such as a (partly) implanted sustained delivery system. Skilled person will understand that such a sustained delivery system may comprise a reservoir for holding the agent as taught herein, a pump and infusion means (e.g., a tubing system).

### EXAMPLES

### 1. TEM and SHV Beta-Lactamases Have Inherent Structural Weaknesses That Predispose Them to Misfolding and Aggregation

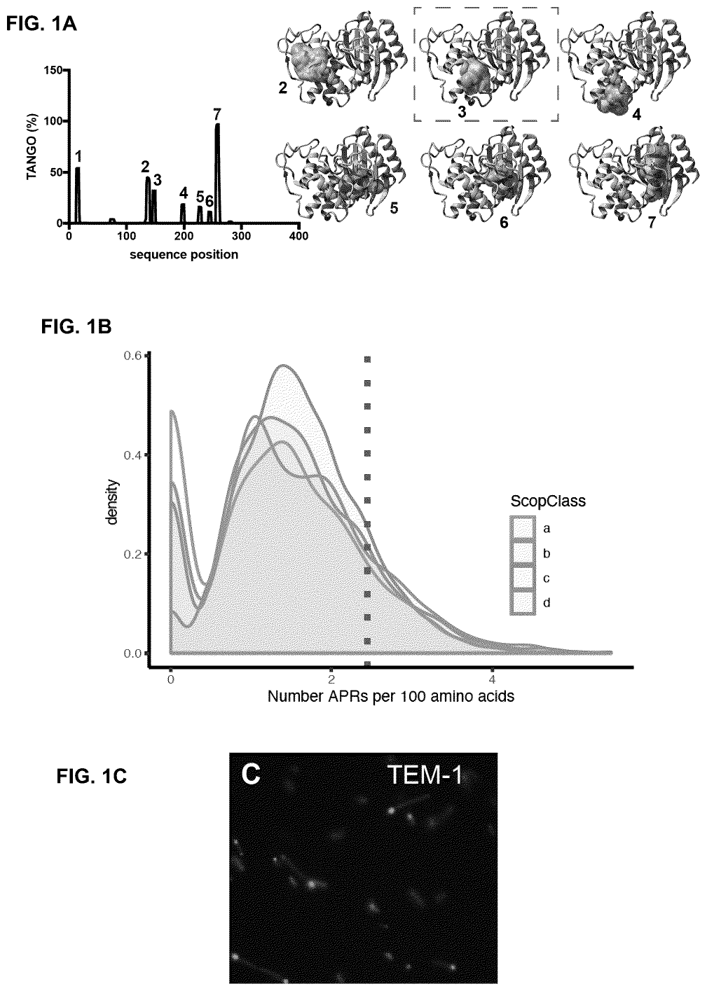

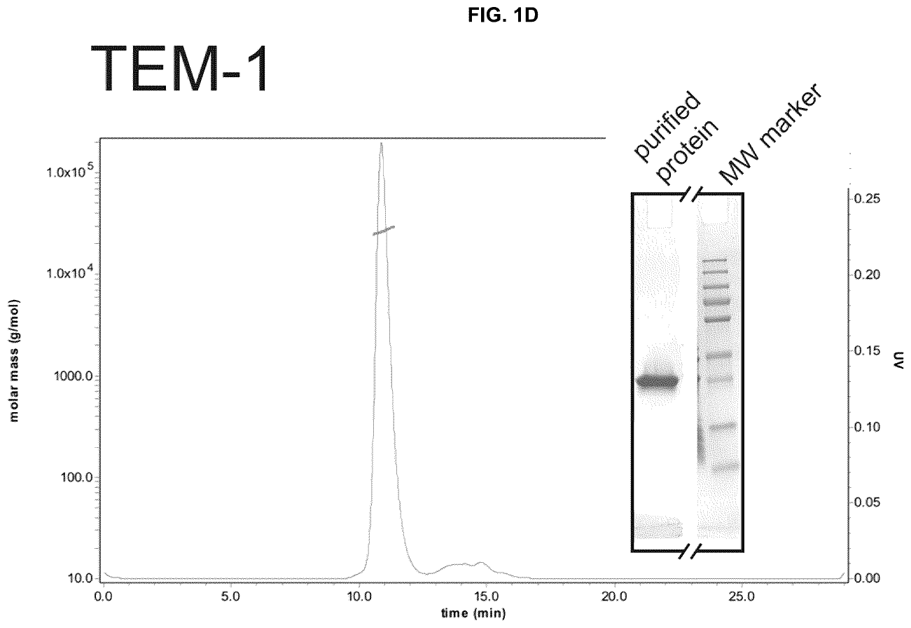

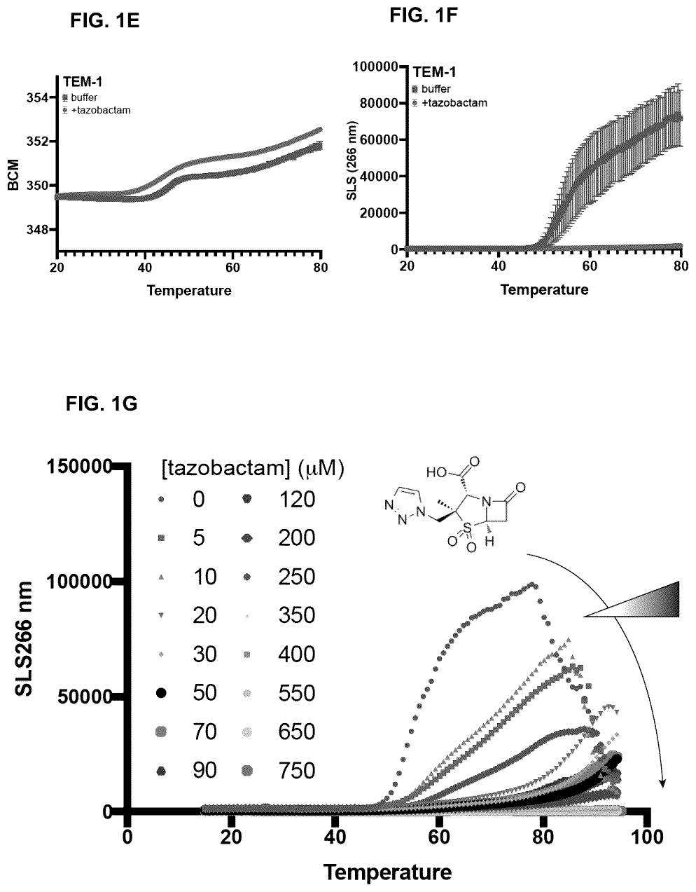

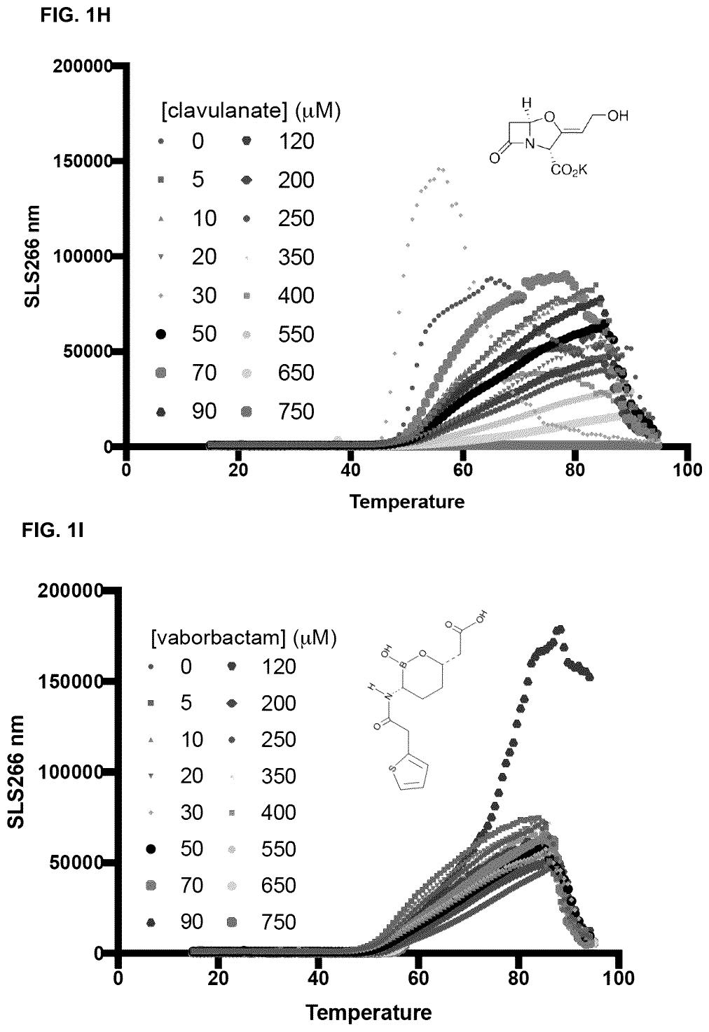

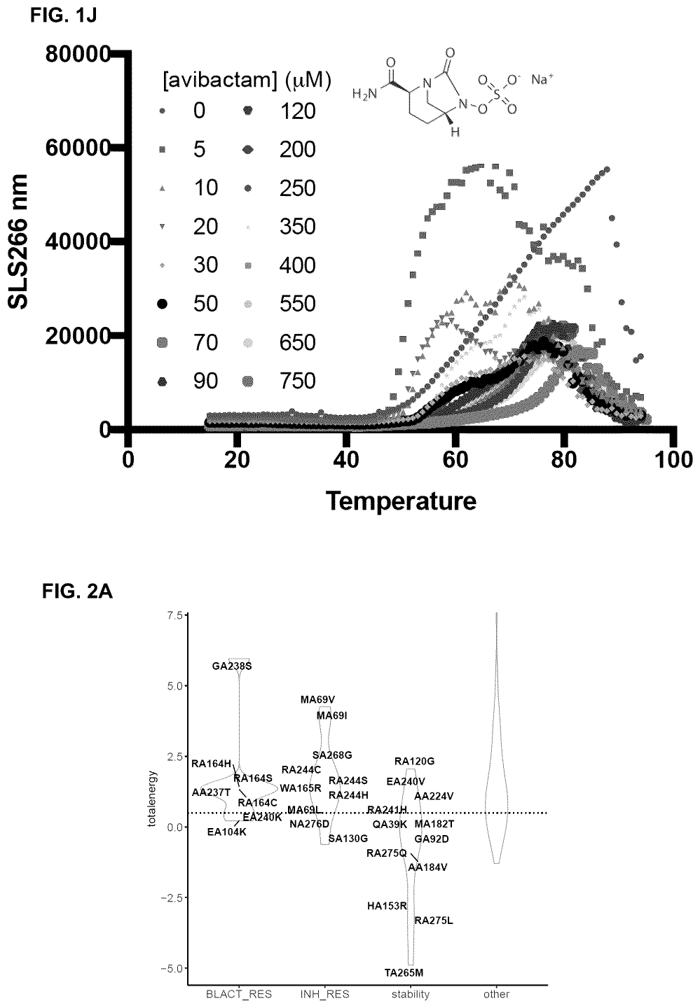

In this example we employed the TANGO algorithm³³ to identify all the APRs in the polypeptide sequence of the TEM-1 beta-lactamase. This led to the identification of 7 candidate APRs, ranging in TANGO aggregation score from 11 to 79%, of which one occurs in the signal peptide and 6 occur throughout the globular part of the protein (FIG. 1A, Table 1). To compare this with the distribution of the number of APRs throughout the different structural fold classes, we turned to the SCOPe database (release 2.06³⁴) and filtered for single chain globular domains and 40% sequence identity using the CD-hit algorithm³⁵. This yielded a dataset of 9017 PDB structures of single protein domains divided into 4 roughly equal fold classes: all alpha helical domains, all beta sheet domains, domains in which alpha-helix and beta sheet are intermixed in the sequence and finally, domains with alpha helix and beta sheet separated in sequence. This analysis showed that six APRs is a high number for a protein of this length, although the value is still well within the tail of the distribution (FIG. 1B). Consistent with a high intrinsic aggregation propensity, we observed spontaneous aggregation of the protein in a typical pattern of polar inclusion bodies when we fused TEM-1 to GFP using a linker sequence and expressed it in E. coli at 37° C. under an arabinose-responsive promotor (FIG. 1C). When we repeated the same with the homologous SHV-11 enzyme, we made a similar observation. We recombinantly purified both proteins (without the GFP tag) after overnight expression at 20° C. in E. coli BL21 using a pET expression system (FIG. 1D). Then, we performed a thermal denaturation whilst simultaneously monitoring the intrinsic fluorescence as a measure of the folding status of the molecule (FIG. 1E), and the static light scattering as a measure of aggregation (FIG. 1F). This revealed that TEM-1 is only marginally stable, with a melting temperature (Tm) of 43.3° C., whereas SHV-11 has a Tm that is much higher, at 68.8° C. However, both proteins show a similarly low aggregation onset temperature (Tagg of TEM-1 and SHV-11 is 44.0° C. and 45.6° C., respectively), consistent with the spontaneous inclusion body formation observed in cells and an overall high aggregation propensity. We confirmed that this did not result from a poor state of the purified material at the onset of the experiment using Size-Exclusion Chromatography (S75, GE Healthcare) coupled to Multiple Angle Light Scattering (SEC-MALS, Wyatt), which showed a single symmetric peak for each protein with a molecular weight that matches that of the monomeric protein within the experimental error of the method (FIG. 1DB). To test if the widely used beta-lactamase inhibitor tazobactam could have the side effect to act as a pharmacological chaperone to SHV-11 and TEM-1, we performed the thermal denaturation of TEM-1 and SHV-11 in the presence of an excess of tazobactam and indeed found an increase in the aggregation onset temperature Tagg, that was more marked for TEM-1 than SHV-11, consistent with the inhibitor acting as a pharmacological chaperone (FIGS. 1I & J). This confirmed that the presence of the inhibitor under appropriate conditions can improve the folding of the enzyme, similarly to the effect of the alpha-galactosidase inhibitor DGJ described above. To probe how general this effect is, we focused on TEM-1, and performed dose-response experiments for the beta-lactam based beta-lactamase inhibitors tazobactam (FIG. 1G) and clavulanate (FIG. 1H), but also the non-beta-lactam beta-lactamase inhibitors vaborbactam (FIG. 1I) and avibactam (FIG. 1J). Although the size and structure of these inhibitors differs widely, all the molecules clearly reduce the aggregation of the enzyme in a dose-responsive manner. Correlation analysis between the amplitude of the static light scattering amplitude at 60° C. and the concentration of the inhibitors shows that the relation is statistically significant (p-val of the correlation being <0.0001, <0.0004, <0.04 and <0.006 for tazobactam, clavulanate, vaborbactam and avibactam, respectively). These results show that the general concept of the pharmacological chaperone effect of beta-lactamase inhibitors is sound, although its magnitude can be expected differ with the many pairs of beta-lactamase/inhibitor, the systematic testing of which falls outside the scope of the current study.

### 2. APRs Have Been Invariant During TEM Evolution

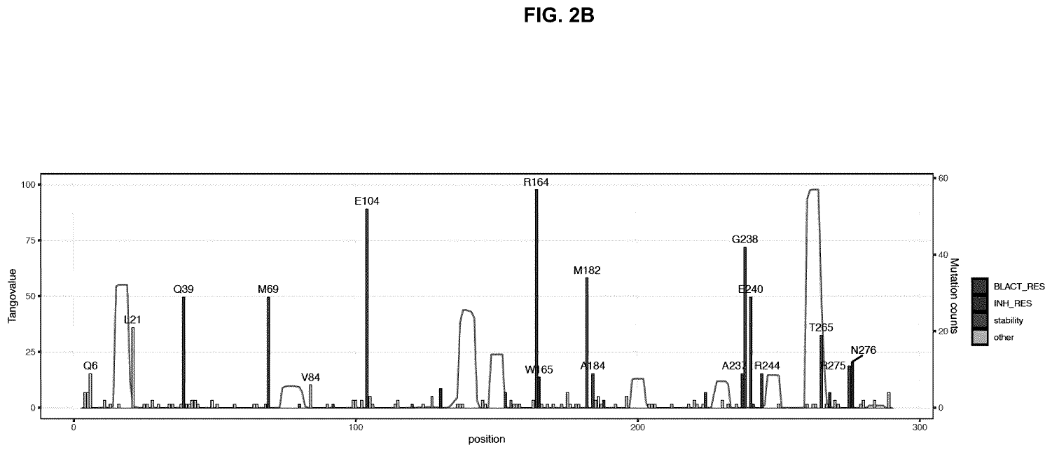

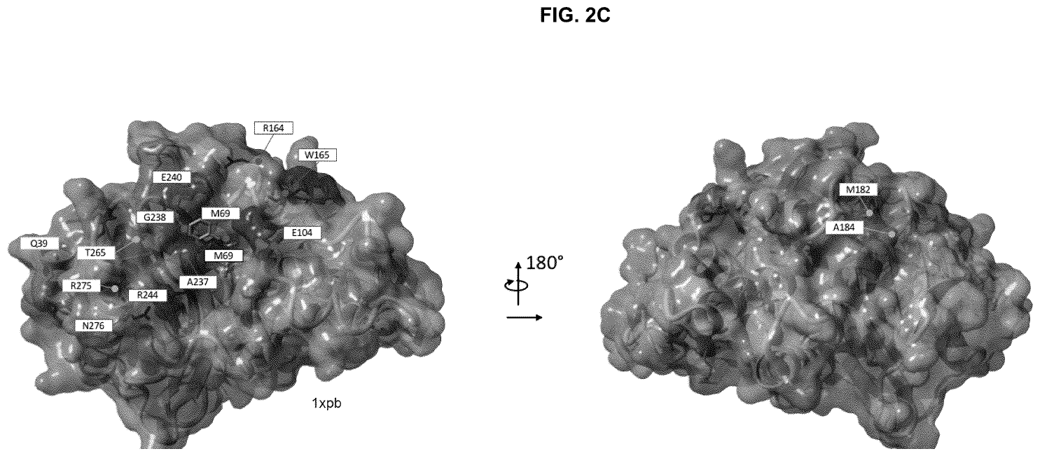

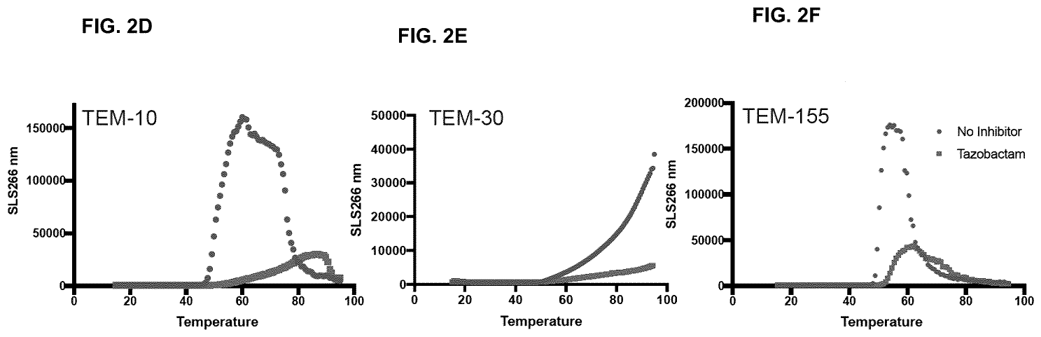

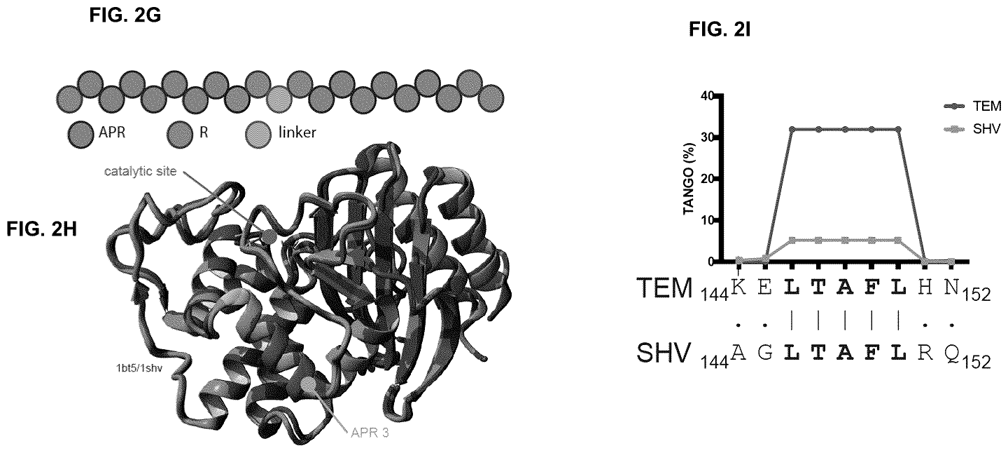

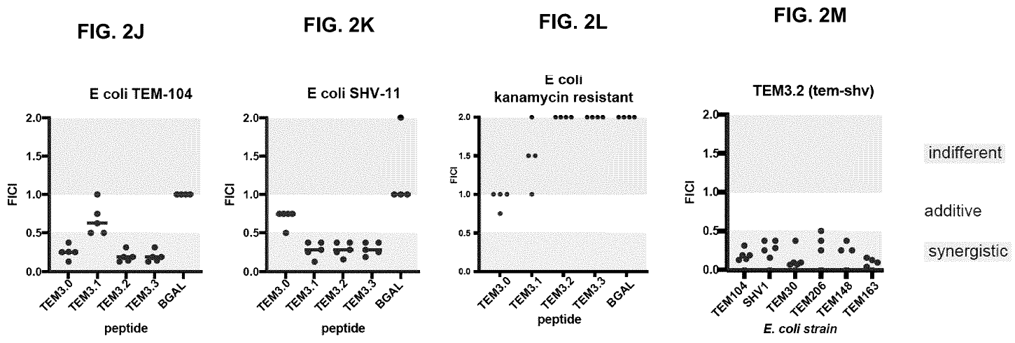

We sought to analyze the effects of mutations frequently found in extended-spectrum TEM variants on the enzyme's stability and aggregation propensity. To do so, we retrieved the TEM variants reported in the Beta-lactamase database (BLDB)³⁶ and from this set parsed all the mutations identified in clinical samples. We then analyzed their effects on TEM stability using FoldX (pdb-structure 1xpb) and mapped their location to that of the aggregation-prone regions as predicted by TANGO. We also cross-referenced these mutations with earlier published work regarding their effects on TEM enzymatic activity and stability^{37,38}. The results of this analysis are shown in FIGS. 2A, B and C. As is clear from FIGS. 2A & 2C, most mutations that extend the TEM spectrum or increase resistance to an inhibitor occur around the substrate-binding site and are destabilizing to protein structure. For example, G238S and R164H, two mutations that are very commonly found in clinical samples and are considered to be driver mutations for TEM spectrum extension, are particularly destabilizing to TEM structure. Several compensatory mutations for these destabilizing effects have been described in literature³⁸, and most of these are indeed predicted by FoldX to enhance protein stability. Strikingly, the lion-share of the mutations observed in clinical samples of extended-spectrum TEM occur outside of the APRs (FIG. 2B). In fact, previous work has revealed that indeed, the highly evolvable regions in the TEM protein are mostly contained within the flexible loops³⁹, away from the APRs in the core. It is clear from these observations that the emergence of destabilizing spectrum-expanding mutations in an already marginally stable protein comes with an evolutionary pressure to introduce compensatory stabilizing mutations. Furthermore, likely owing to this marginal stability, the positioning of these mutations seems to be limited to flexible loops and surface positions in the protein, while APRs are largely untouched. As such, we reasoned that inducing aggregation of ESBLs through their largely immutable APRs is a viable knock-down approach that cannot be easily escaped through random mutations that are compatible with enzymatic function.

Of note, the number of amino acid substitutions that separate the vast majority of TEM variants from the original TEM-1 sequence is 4 or less out of 263 residues, corresponding to a sequence identity of >98%. Therefore, although these mutations have a major impact on the enzymatic activity because the active site consists of only a few residues, the mutations are not likely sufficient to significantly modify the overall folding and aggregation behaviour of the protein. Hence, in order to develop a sense if the intrinsic aggregation observed for TEM-1, as well as the molecular chaperoning effects of the beta-lactamase inhibitors also hold for other TEM variants, we selected representative variants for experimental studies, ensuring to sample the TEM sequence space by including key mutations (highlighted in FIG. 2B). TEM15 (=TEM1^{R2445}), TEM30 (=TEM1^{E104K; G2385}) and TEM52 (=TEM1^{E104K; G2385; M182T}) harbor some of the most common active site modifying mutations found in ESBLs, including the stabilizing mutation M182T. TEM10 (=TEM1^{R1655; E240K}) has been implicated in outbreaks across the US and Europe and TEM155 (=TEM1^{Q39K; R164S; E240K}) was included as a more recent extended spectrum, inhibitor-resistant BL. All variants were recombinantly produced as before and the aggregation was assayed in the presence and absence of tazobactam (FIG. 2D-F). These results lead us to conclude that in the recent evolution of the TEM enzyme, it has retained its intrinsic aggregation propensity, and the pharmacological chaperone effect of tazobactam thereon.

### 3. Design and Identification of Peptides That Inactive TEM-1

In order to generate a synthetic amyloid peptide capable of inactivating the TEM-1 beta-lactamase enzyme by capitalizing on its aggregation propensity, we applied a previously developed design pattern for synthetic aggregating sequences. This involves a tandem repeat of the APR, each instance flanked by charged amino acids for solubility and separated by a short peptide linker^{19,20,23} (FIG. 2G). Previous work has shown that good bacterial uptake can be achieved using one positively charged arginine residue on the N-terminal side of each APR in the tandem and two arginine residues on the C-terminal side. This has the additional benefit of reducing the self-aggregation of the peptides and increasing their solubility prior to target engagement. As before, we employed a single Pro residue as a linker between the APRs and focused on an APR length of seven, extending shorter APRs to this length by incorporating flanking amino acids unless they were obvious aggregation breakers (Arg, Lys, Asp, Glu or Pro, see Table 1). We obtained these peptides from solid phase synthesis, followed by HPLC purification to a purity judged by reverse phase chromatography of at least 90% (Genscript). To screen these peptides for their ability to inhibit beta-lactamase activity, we used a clinical isolate of E. coli (isolated at University Hospitals Leuven, called UZ_TEM104), which we verified to carry the TEM β-lactamase gene by qPCR and Sanger sequencing and that is highly resistant to penicillin G, showing a Minimal Inhibitory Concentration (MIC) as high as 1600 μg/mL (Table 2). We determined the MIC of the peptides against this E. coli strain and found that they by themselves were not toxic with a MIC up to 100 μg/ml (Table 2). Then, for this strain, we determined the MIC of penicillin G in the presence of the peptides at a fixed concentration of 50 μg/mL (Table 2) and found that the MIC dropped, particularly in the presence of the peptide called TEM3.0 (based on APR 3, RLTAFLHNRRPRLTAFLHNRR (SEQ ID NO: 5)), with an 8-fold reduction of the MIC of penicillin G. When we added the widely used β-lactamase inhibitor tazobactam⁶ at 50 g/mL, it reduced the MIC of penicillin G by 4-fold. As expected from the different modes of action of tazobactam and peptide TEM3.0, their combined effect (25 μg/ml of each) reduced the MIC for penicillin G further (to below 50 μg/mL) (Table 1).

### 4. Cross-Reactivity With SHV Beta-Lactamase

Interestingly, peptide TEM3.0 is based on a 5-mer APR (LTAFL (SEQ ID NO: 11), TANGO=32), which in the peptide has been extended to 7 (LTAFLHN (SEQ ID NO: 9)) but that is conserved in all 227 TEM sequences in the beta-lactam database⁴ and the 7-mer sequence is conserved in 224 out of them (3 carry the H to R mutation in position 6), suggesting that all strains that derive their beta-lactam resistance from TEM1 should be sensitive to treatment with peptide TEM3.0. Moreover, the core sequence of peptide TEM3.0 also occurs in the structurally related SHV β-lactamase (FIG. 2E). Although the C-terminal extension residues H and N have been replaced with physicochemically similar R and Q, respectively (FIG. 2H). Hence, to find a peptide with cross-reactivity between the closely related TEM and SHV, we generated TEM3.1, TEM3.2 and TEM3.3, by replacing both or either one of the APR repeats with the version found in SHV (yielding TEM 3.1 RLTAFLRQRRPRLTAFLRQRR (SEQ ID NO: 6), TEM 3.2 RLTAFLHNRRPRLTAFLRQRR (SEQ ID NO: 7)and TEM 3.3 RLTAFLRQRRPRLTAFLHNRR (SEQ ID NO: 8), respectively). To quantify the synergy between these four peptides and penicillin G, we performed a so-called checkerboard assay, which allows to compare the activity of two test compounds co-incubated (here peptide and a beta-lactam antibiotic), to their individual activities in isolation. The result from the assay is quantified as the Fractional Inhibitory Concentration Index (FICI), and it is generally accepted that values below 0.5 indicate synergistic effects between the compounds. As a control, we generated a peptide using the same design, but based on a previously identified APR from the β-galactosidase enzyme (BGAL)¹⁶ from E. coli (APR=SVIIWSL, peptide sequence=RSVIIWSLGRRPRSVIIWSLGRR) that has a MIC value>100 μg/mL on E. coli strain UZ_TEM104. We evaluated the synergy of the peptides with penicillin G in clinical E. coli isolates carrying the TEM (FIG. 2I) or SHV enzymes (FIG. 2J). The TEM3.0 peptide, which contains the TEM-version of the APR in both repeats of the tandem design, showed clear synergy only in the strain containing the TEM1 enzyme, but not in a strain containing the SHV-11 enzyme. For TEM3.1, which contains the SHV-11 version of the APR in both repeats, showed the opposite: a clear synergy in a strain containing SHV-11, but not in a strain containing TEM1. The TEM3.2 and TEM3.3 peptides, that contain both the TEM and the SHV APRs, showed synergy in both strains containing SHV-11 and TEM-1, suggesting a perfect matching sequence is required for efficient interaction between peptide and protein target. In line with this the BGAL control peptide showed no synergy with penicillin G, as expected (FIGS. 2J & K), also, mutant peptides in which proline residues were introduced in the APR regions to break the beta-interactions did not show synergy. As a further control, we also performed a checkerboard assay with an E. coli strain that was resistant to the aminoglycoside kanamycin and observed no synergy between this unrelated antibiotic and our peptides (FIG. 2L), in line with the notion that we are inhibiting a beta-lactamase but not the aminoglycoside degrading enzyme. Then, we further compared the activity of the peptides in 16 additional E. coli clinical isolates containing TEM or SHV enzymes (Table 3), by evaluating the MIC of ampicillin in the presence of a fixed concentration of 30 μg/ml of TEM3.2. Overall, we found that 87% (14 out of 16) of the clinical isolates containing TEM or SHV were sensitized by the presence of 30 μg/mL TEM3.2 to ampicillin. The list of strains included some that harbored only TEM or SHV, but others contained additional Ambler Class A (CTX-M or OXA) or Class B (VIM and NDM) beta-lactamases. The CTX-M class of beta-lactamases is a structural homolog to TEM and SHV, and from these data appears to be sensitive to the peptide treatment. The presence of CTX-M and OXA did not appear to modify the effect of the peptide significantly, which may be related to the lower turnover rate of these carbapenemases for ampicillin used in these experiments⁴⁰⁻⁴³, but may to some extent also result from indirect effects on their folding and expression due to the proteotoxic stress resulting from the TEM/SHV aggregation. In contrast, the presence of the NDM-type metalloproteases, that does have a high affinity for Ampicillin and that is completely distinct in sequence and structure from TEM and SHV, seemed to completely prevent the effect of the peptide on the beta-lactam sensitivity of the strains in which it occurred (see also further). As it appeared that TEM3.2 had activity in most isolates tested, we decided to focus further analysis on this peptide. Furthermore, we tested the performance of the TEM3.2 peptide in a checkerboard assay on strains carrying ESBL mutants of TEM, i.e. extended-spectrum mutations that cluster around the active site of the enzyme, and not in the aggregation prone region targeted by the peptide, and as expected from our mode of action, we observed synergy with ampicillin on these strains (FIG. 2M).

### 5. The Peptide Causes Aggregation of the Target Protein

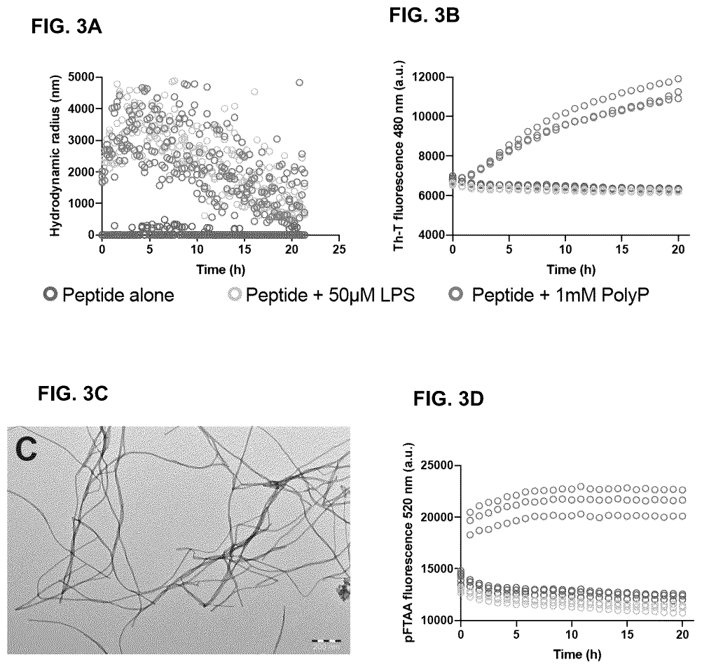

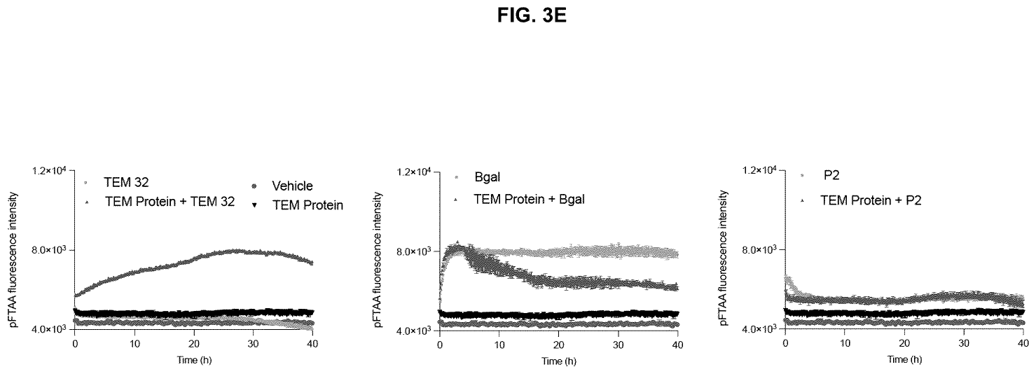

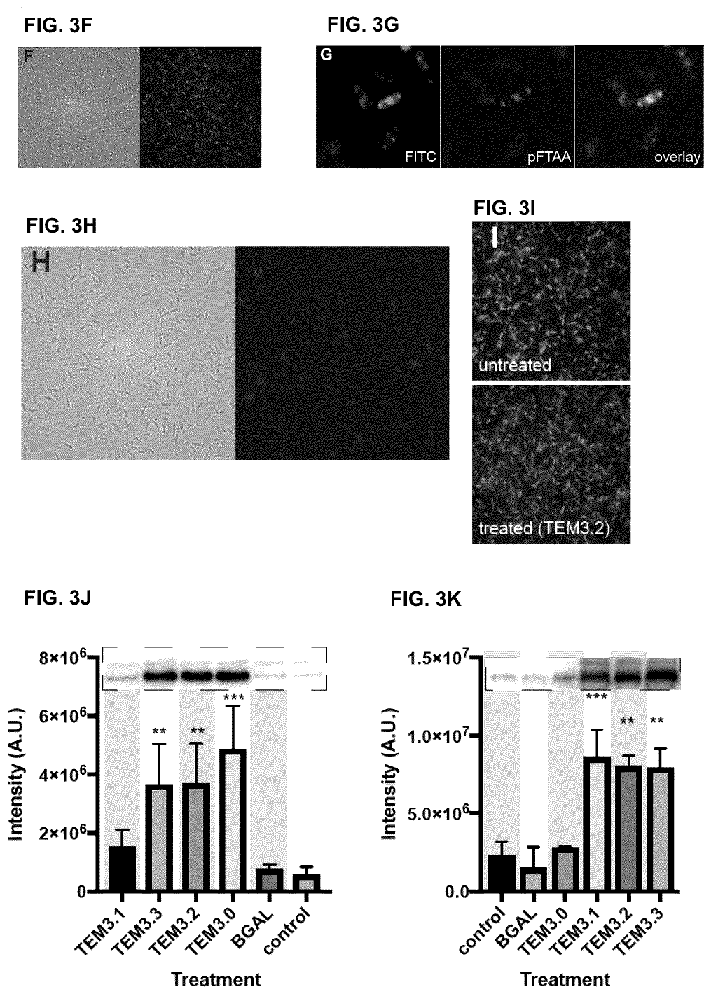

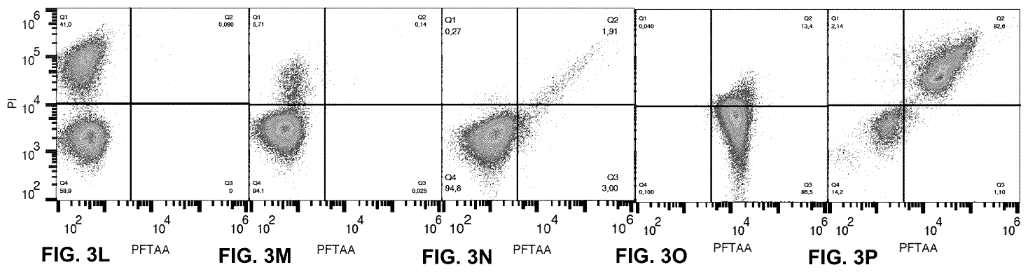

As expected from the balanced design between aggregation propensity and charge, Dynamic Light Scattering (DLS) showed that the peptide is soluble in vitro, but aggregates readily in the presence of poly-ionic counterions that are found abundantly inside bacterial cells such as polyphosphate (PolyP) (FIG. 3A), a known modifier of amyloid formation⁴⁴ that is upregulated in times of (proteotoxic) stress. Thioflavin T (Tht) binding (FIG. 3B) and Transmission Electron Microscopy (TEM, FIG. 3C, 20 h incubation at RT) revealed amyloid-like aggregation in the presence of polyphosphate. These fibrils also stain positive for pentameric formyl thiophene acetic acid (pFTAA), an extensively characterized amyloid-specific dye⁴⁵⁻⁴⁷, that specifically binds to amyloid-like aggregates as well as disease-associated protein inclusion bodies 47 (FIG. 3D). Aggregation-induction by polyP was dose-dependent and could also be induced using agents such as Poly-Ethylene Glycol (PEG) that mimic the molecular crowding that the peptide will encounter in a cellular environment. When we evaluated the effect of peptide 3 on the aggregation of recombinantly purified TEM beta-lactamase protein using the thioflavin-T aggregation assay, we observed that the peptide was a potent inducer of the aggregation of the TEM betalactamase (FIG. 3E). This effect was specific as it could not be induced with a Pept-In with a different APR (ColPeptin⁴⁸).

Structured Illumination Microscopy (SIM) images of E. coli UZ_TEM104 treated with a FITC labelled version of TEM3.2 showed uptake of the peptide into the bacterial cytosol, where it is located in inclusion bodies, the defining organelles of aggregation, that also stain positive for pFTAA (FIGS. 3F & G). When we performed the same experiment with an E. coli strain lacking TEM (ATCC 25922), we observed only very limited inclusion body formation (FIG. 3H, compare to F), showing that this effect is dependent on the presence of the target protein. Moreover, when we treated bacterial strains stably expressing GFP (FIG. 3I) or proteins fused to FPs with TEM3.2, we did not observe any induced aggregation of the fluorescent proteins, in agreement with the specific induction of aggregation of the TEM beta-lactamase by this Pept-in. In a western blot using a TEM-specific monoclonal antibody of the inclusion body (IB) fraction prepared from E. coli UZ_TEM104, treated with the four TEM peptides, as well as the unrelated BGAL peptide and a buffer control, we observed a clear enrichment of the TEM protein in IBs after treatment with the TEM3.0 (TEM-TEM configuration of APRs), TEM3.2 and TEM3.3 (mixed TEM-SHV APRs), but not for TEM3.1 (SHV-SHV configuration) (FIG. 3J), consistent with the aggregation of the targeted enzyme as intended by the design. The full blot and control lanes with only peptide or recombinant protein clearly show that this band is protein-specific and that there is no cross-binding of the antibody to the peptide. Moreover, we confirmed the presence of the protein in the correct band using mass spectrometry proteomics. We repeated this with a strain expressing SHV-154, blotting with a polyclonal rabbit antibody raised against the recombinant SHV enzyme described above (FIG. 3K). We found that the SHV enzyme accumulates in IBs in response to treatment with TEM3.1, TEM3.2 and TEM3.3, but not TEM3.0, in accordance with the APR configuration in these peptides and reflecting nicely the synergy data mentioned above.

We confirmed these data using FACS analysis of E. coli UZ_TEM104 in which we monitored two fluorescence channels: one for Propidium Iodide (PI), a cell death stain that enters cells with an impaired cell wall permeability, and the second for pFTAA, to monitor aggregation in the bacteria. First, we tested the assay using a mixture of live and heat inactivated (FIG. 3L) or just live bacteria (FIG. 3M), showing that heat inactivated, but not live bacteria are stained with PI, and neither are stained with pFTAA. When we treated bacteria with 400 μg/mL penicillin G only, we observed no major change in either channel (FIG. 3N), consistent with a resistant strain that survives the treatment. When we treated with 50 μg/mL peptide TEM3.2, we observed a shift of the bacterial population only in the pFTAA channel, consistent with non-lethal aggregation occurring in >99% of the cells (FIG. 3O). Finally, when we treated with both 400 μg/mL penicillin G and 50 μg/mL peptide TEM3.2, we observed a shift in both channels, showing the cell death depends on the presence of both penicillin G and TEM3.2 (FIG. 3P).

### 6. Efficacy of TEM3.2 in the Treatment of Urinary Tract Infection in Mouse

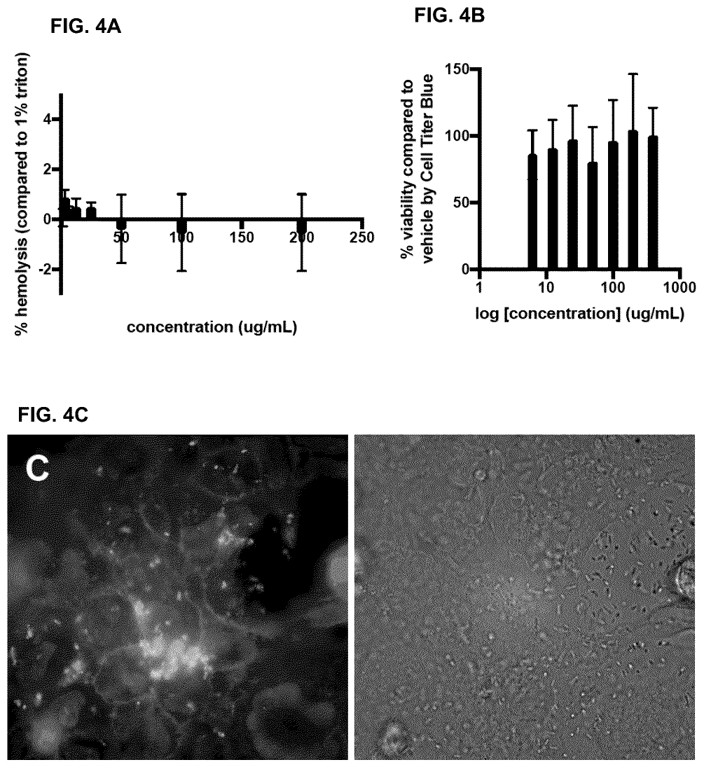

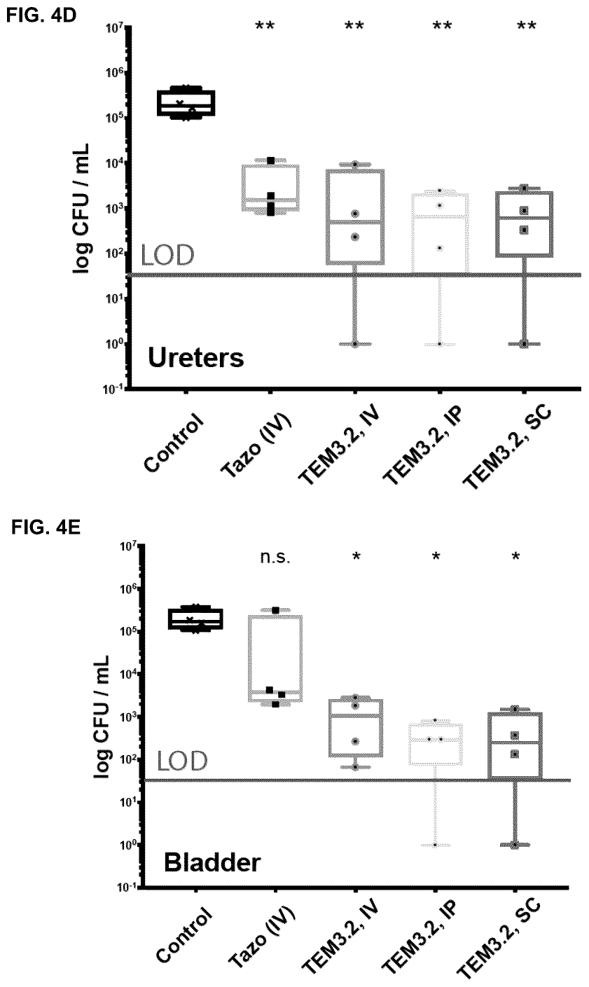

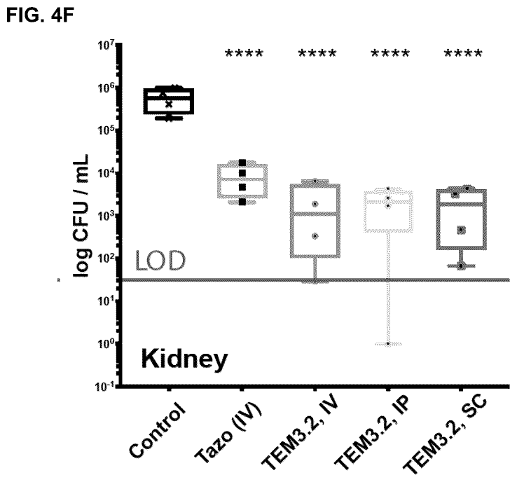

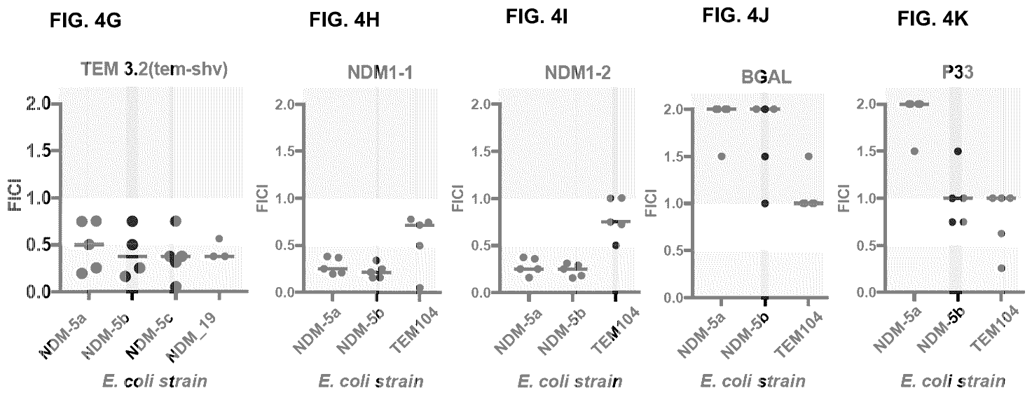

Given this broad activity on clinical isolates, we wondered if the TEM3.2 peptide could be used to re-sensitize bacteria to ampicillin treatment in vivo, in a mouse Urinary Tract Infection (UTI) model. Prior to this, we wanted to assess the potential of the designed peptides to induce off-target aggregation of mammalian proteins, and we searched the entire human proteome from UniProtKB/Swiss-Prot database (release 2020_04) and found that none of the 20,359 proteins contain an exact match to any of the targeted APRs under study. The same conclusion was reached for the mouse proteome. Then, we established that the peptide was not hemolytic to human erythrocytes (from healthy volunteer donors, FIG. 4A) and not cytotoxic using a Cell Titer Blue assay to human cell lines (in HEK293, HeLa, NCI-H441 and SH-SY5Y), as well as primary Human Umbilical Vein Endothelial Cells (HUVEC) and primary mouse cortical neurons (FIG. 4B). We did not observe toxicity of the peptide to mammalian cells in this manner. Moreover, when we treated co-cultures of human cell lines and E. coli TEM1 with a FITC-labelled derivative of peptide TEM3.2, we observed the fluorescence in the bacterial but not the mammalian cells using fluorescence microscopy, suggesting preferential uptake into the bacteria (FIG. 4C).

We turned to a FITC-labelled derivative of peptide TEM3.2 for the in vivo studies, reasoning that this would allow us to monitor if the peptide could reach the bladder upon parenteral administration using fluorescence imaging, and this was confirmed in a limited study. We then extracted urine from the treated animals (time) and performed SDS-PAGE analysis which showed the peptide to be largely intact at this stage, allowing us to follow the FITC label. We also performed a tolerability study using a dose escalation method by administering the peptide at 2, 5, 10, 15 and 20 mg/kg via different parenteral routes: intravenous (IV), intraperitoneal (IP) and subcutaneous (SC) in female C57BL/6JAX mice, aged 8-10 weeks. We recorded any clinical signs by looking at their activity, posture and respiration rate, to establish the tolerance of the animals to the substance, and found no obvious signs of toxicity. As a final step, we exposed animals to daily IV injection of TEM3.2 (5 mg/kg) and performed a hematologic analysis, which showed no major discrepancies to vehicle treated controls. Then we used a catheter to inoculate the urethra of female C57BL/6JAX mice of 8-10 weeks with 1×10⁸ cells of a uro-pathogenic E. coli strain (UPEC strain blaTEM-1, MIC_{Ampicillin}=1200 μg/mL, which in the presence of either 32 μg/ml of either TEM3.2 or tazobactam drops to 25 μg/mL). At 60- and 120-minutes post-inoculation, the mice received 30 mg/kg of ampicillin, plus 10 mg/kg of FITC-TEM32 administered IV, IP or SC, or 10 mg/kg tazobactam, administered IV, as a control. Vehicle alone (0.9% NaCl) was also administered (IV). After 24 h the animals were sacrificed and the bacterial load in the bladder, kidney and ureter was quantified by determining the number of Colony Forming Units (CFU) per mL of tissue extract (FIGS. 4D, E and F). These graphs showed a reduction of the bacterial load of 2 log folds (up to 2.8), that was most robust in kidney and ureter, and that was slightly outperforming tazobactam (best reduction 1.8 log folds). When we performed FACS analysis similar to FIG. 3 on the bacteria from treated, infect animals, we could detect both peptide uptake and protein aggregation in these bacteria, consistent with a conservation of our mode of action to the in vivo situation. These data provide proof of concept that the molecules are capable of resensitizing this strain of beta-lactamase carrying E. coli to the beta-lactam antibiotic ampicillin in vivo, which may lead to actual therapeutic applications.

### 7. Targeting the NDM1 Beta-Lactamase

The sensitivity of clinical strains to the TEM3.2 peptide seemed to depend on the presence of other resistance factors since strains carrying the New Delhi Metalloprotease (NDM1) beta-lactamase, which bears no structural or sequence similarity to TEM/SHV, did not respond to TEM3.2 (Table 3). Indeed, when we analyzed TEM3.2 in a checkerboard assay against E. coli strains carrying NDMs, we found little or no synergy against strains containing NDMs, although we observed significant spread (FIG. 4G). As we wondered about the generality of our peptide design approach, we set out to design a peptide to inactivate a structurally unrelated beta-lactamase. To this end we turned to the Ambler class B beta-lactamase NDM-1 that confers resistance to many beta-lactam antibiotics, including carbapenems, and strains carrying this enzyme was soon dubbed ‘superbugs’ that are only partially sensitive to colistin and tigecycline⁴⁹. This enzyme was first isolated from a Swedish patient of Indian descent in 200850, by 2010 could be detected in clinical isolates throughout the UK and India⁵¹ and by 2015 was detected in over 70 countries worldwide⁵². We set up a screen similar to the one executed for TEM described above, leading to the identification of 2 peptides named NDM1-1 (RTAQILNWRRPRTAQILNWRR, (SEQ ID NO: 12)) and NDM1-2 (RLAAALMLRRPRAQILNWIRR, (SEQ ID NO: 13)) that target the T₁₀₁AQILNW₁₀₇ APR ((SEQ ID NO: 14), detected by the Waltz algorithm⁵³, located in an exposed alpha-helix in the native structure. Both peptides show synergy in strains containing NDM5a and NDM5b (FIG. 4H and I for NDM1-1 and NDM1-2, respectively), but not in a strain containing TEM-1. As controls, we tested a non-toxic peptide on both strains (BGAL, FIG. 4J) as well a toxic peptide (P33, FIG. 4K), and neither showed synergy on any of the strains used. These results show that targeting beta-lactamases for aggregation could have a broader applicability.

### Materials and Methods

### Bioinformatics Analysis

Protein sequences for beta-lactamase TEM, SHV and NDM bacterial strains were obtained from UniProt⁵⁵. We employed the software algorithm TANGO33 to identify APRs across this work, using a score of 5 per residue as the lower threshold and a parameter configuration of temperature at 298K, pH at 7.5, and ionic strength at 0.05 M.

### TEM Variant Analysis

Known TEM variants were retrieved from the Beta-Lactamase DataBase (BLDB)⁴. The mutations found in these variants were cross-referenced with literature to classify them according to their observed effects: offering resistance to an extended spectrum of β-lactams, offering resistance to inhibitors, stabilizing the TEM structure or other^{8,56,57}. The effect of each mutant on protein stability was predicted through the FoldX forcefield⁵⁸. To this end, PDB-structure 1xpb⁵⁹ was first energy-minimized using the FoldX RepairPDB command, and subsequently the effect on stability of individual mutations was assessed using the BuildModel command, with default settings. As stated above, the TEM sequence was further analysed using the TANGO aggregation prediction software, using default settings. The mutated residues were visualised in the TEM structure using YASARA⁶⁰.

### Peptides Design and Synthesis

Peptide hits were ordered from Genscript at >90% purity and were also produced in-house using the Intavis Multipep RSi automated synthesizer using solid phase peptide synthesis. After synthesis, crude peptides were stored as dry ether precipitates at −20° C. Stock solutions of each peptide were either prepared in 100% DMSO (only for initial screening assays) or following the optimized protocol: peptides were dissolved in 1 M NH₄OH, allowed to dissolve for ˜5 minutes, and dried in 1.0 mL glass vials with a N₂ stream to form a peptide film. This film was dissolved in buffer containing 50 mM Tris (pH 8.0) and 20 mM guanidine thiocyanate. Peptides were N-terminally acetylated and C-terminally amidated.

### Biophysics Study

A DynaPro DLS plate reader instrument (Wyatt, Santa Barbara, CA, USA) equipped with an 830 nm laser source was used to determine the hydrodynamic radius (RH) of the peptide particles. Two hundred microliters of each sample (at 100 or 10 μM, unless stated otherwise) were placed into a flat-bottom 96-well microclear plate (Greiner, Frickenhausen, Germany). The autocorrelation of scattered light intensity at a 32° angle was recorded for 5 s and averaged over 20 recordings to obtain a single data point. The Wyatt Dynamics v7.1 software was used to calculate the hydrodynamic radius by assuming linear particles. The amyloid-specific dye Thioflavin-T (Th-T, Sigma-Aldrich, CAS number 2390-54-7) was used to study the aggregation state of peptides. Two hundred microliters of each peptide sample (at 100 [symbol] M, unless stated otherwise) was placed into a flat-bottom 96-well microclear plate (Greiner, Frickenhausen, Germany) and the dye was added to a final concentration of 25 μM. A ClarioStar plate reader (BMG Labtech, Germany) was used to measure fluorescence by exciting the samples at 440-10 nm and fluorescence emission was observed at 480-10 nm (or a complete spectrum ranging from 470 nm-600 nm). Aggregation kinetics were obtained by placing 200 μl of the peptide solution with a final concentration of 25 μM thioflavin-T (Th-T) into a flat-bottom 96-well microclear plate. Fluorescence emission was monitored at 480-10 nm after excitation at 440-10 nm. Every 5 min Th-T fluorescence was measured.

### Bacterial Collection and Growth Conditions

Beta-lactamase clinical samples were collected from University Hospitals Leuven and tested for ESBL production using the disk diffusion method⁶¹. The beta-lactamase reference isolates were purchased from IHMA International Health management associates. Bacterial strains were cultivated in Mueller Hinton Broth (MHB, Difco) at 37° C. Whenever required growth media were supplemented with appropriate antibiotic to the medium or plates (kanamycin 30 μg/mL, L-arabinose 0.5 mg/mL, and IPTG 1 mM/mL). Escherichia coli BL21 (Thermo Fisher Scientific, Belgium) was used for cloning and plasmid amplification. For selection of antibiotic resistance colonies, E. coli carrying plasmids was grown in LB agar plates supplemented with the relevant antibiotic. Bacterial CFU counting was done on blood agar plates (BD Biosciences, Belgium) or MHA agar plates. Species identification and antibiograms for all clinical isolates were performed using MALDI-Tof and VITEK® 2 automated system (BioMérieux, France). All strains used for this study together with their resistance profile are listed in Table 4.

### In Vitro Toxicity on the Mammalian Cells

The Cell Titer Blue assay was performed to evaluate the cell viability according to the instructions of the manufacturer (Promega, USA). The peptide treatments were done in DMEM medium without serum. Briefly, cells were seeded to approximately 20.000 Hela cells per well in a 96-well flat-bottom plate (BD Biosciences 353075) and incubated at 37° C. with 5% CO2 and 90% humidity. Peptides were diluted in cell medium and cells were treated for 24 hours. 20 μL of the CellTiter Blue reagent was added to each well and the plate was incubated for one hour at 37° C. The fluorescence was measured at 590 nm by exciting at 560 nm with a ClarioStar plate reader (BMG Labtech, Germany).

Hemolytic activity was evaluated by measuring the amount of released hemoglobin. Fresh blood was pooled from healthy volunteers (collected from Rode Kruis Vlaanderen, Mechelen, Belgium). EDTA was used as the anticoagulant. Briefly, erythrocytes were collected by centrifugation 3000×g for 10 min. The cells were washed with phosphate-buffered saline (PBS) several times and diluted to a concentration of 8% in PBS. Hundred microliters of 8% red blood cells solution was mixed with 100 μL of serial dilutions of peptides in PBS buffer in 96-well plates (BD Biosciences, Belgium). The reaction mixtures were incubated for at least 1 h at 37° C. Plate centrifuged for 10 min at 3000×g. The release of hemoglobin was determined by measuring the absorbance of the supernatant at 495 nm. Erythrocytes in 1% Triton and maximum used concentration of buffer were used as the control of 100% and 0% hemolysis, respectively.

### Antibody and Antibiotic Product Codes

The antibodies and antibiotic product codes used are as follows: monoclonal anti-TEM (Abcam, UK ab12251-8A5A10) 0.5 μg/mL, polyclonal rabbit anti-SHV (custom-made by Eurogentec, Belgium) 1 μg/mL, chicken polyclonal anti-beta Galactosidase (Abcam, ab145634 antibody (ab9361) 2 μg/mL. Goat Anti-Mouse IgG HRP secondary antibodies (ab97040); Rabbit Anti-Mouse IgG HRP (ab6728); Goat Anti-Chicken HRP (ab97135).

The antibiotics used for this study: Penicillin G sodium (Benzylpenicillin sodium, Abcam, catalog #ab145634) 1 μg/mL, Ampicillin (Duchefa Biochemie, Netherlands, A0104.0025), tazobactam sodium salt (Sigma-Aldrich, catalog #T2820-10MG), erythromycin, CAS number 114-07-8 (Sigma-Aldrich, catalog #E5389), chloramphenicol, CAS number 56-75-7 (Duchefa Biochemie), and kanamycin CAS number 56-75-7 (Duchefa Biochemie).

### MIC Determination

Determination of MIC values using the broth microdilution in the 96-well plate (BD Biosciences, Belgium) according to the EUCAST guideline, which was performed in 96-well polystyrene flat bottom microtiter plates (BD Biosciences). Briefly, a single colony was inoculated into 5 mL Difco™ Mueller Hinton Broth (BD Biosciences Ref 275730) and grown to the end-exponential growth phase in a shaking incubator at 37° C. Cultures were subsequently diluted to an MacFarland (0.5 optical density) then the culture was diluted to reach 10⁶ CFU/mL in fresh MHB medium. 50 μl of different concentration of peptides ranging from 128 to 2 μg/mL were serially diluted to the sterile 96-well plate in MHB. 50 μL of the diluted bacteria in MHB were pipetted into 96-well plates to reach the final volume of 100 μL. The bacteria grown with the maximum concentration of carrier and medium were considered as positive and negative controls, respectively. The plates were statically incubated overnight at 37° C. to allow bacterial growth. OD was measured at 590 nm a multipurpose ultraviolet-visible plate reader, and the absorbance of the growth bacteria was measured using absorbance reader. Bacterial growth was also visually inspected and agreed well with the OD reading.

### Checkerboard Assay

For the analysis of the synergy between the peptides with other antibiotics, a checkerboard assay was performed. Referring to the MICs of the selected peptides, checkerboard assay was designed to define their FICIs (Fractional Inhibitory Concentration Index) in combinations against different clinical isolates^{62,63}. Briefly, a total volume of 100 μL of Mueller-Hinton broth was distributed into each well of the 96-well plates. The first compound (peptide) of the combination was serially diluted vertically (128, 64, 32, 16, 8, 4, 2, 0 μg/mL) while the other drug (Beta-lactam or Kanamycin) was diluted horizontally in 96 well plate (from 3200 to 3 μg/mL). The total volume of each microtiter well was inoculated with 100 μL of MHB containing 1×10⁶ CFU/mL bacteria. The plates were incubated at 37° C. for 24 h under aerobic conditions without shaking. Calculation of the FICI is used to analyze the results of the checkerboard assay by estimating the degree of synergistic effect. FICI is calculated as the sum of the individual fractional inhibitory concentrations (FICs) for each drug (where MIC A and MIC B denote the MIC of each drug alone, and MIC A_{A+B} and MIC B_{A+B} denote the concentrations of A and B in the drug combination). FICI=(MIC A_{A+B}/MIC A)+(MIC B_{A+B}/MIC B). With FICI≤0.5, the combination of antibiotics considered as a synergistic effect, 0.5<FICI≤1 indicates additivity, FICI>1 indicates indifference.

### Flow Cytometry Analysis

Bacterial cells in the cleaned suspensions were stained with both propidium iodide (PI) and FITC peptide to evaluate the killing rate and peptide uptake in a two-dimensional analysis. Briefly, end-exponential growth phase E. coli cells (10⁶ CFU/mL) washed with PBS and treated with peptides at sub MIC (0.25×MIC) and sub MIC of the Penicillin for several hours at 37° C. Treated bacteria washed with PBS buffer two times. One microliter of PI (Invitrogen) was added to the bacteria and incubated for 5 min. The bacteria were used by FACS tubes for 40000 events. To correlate the activity of the peptides with cell death, the fluorescence intensity was measured in two channels using the Gallios™ Flow Cytometer (Beckman Coulter, USA), PI: excitation 536 nm and emission 617 nm, FITC: excitation 490 nm and emission 525 nm. Heated bacteria at 90° C. for 10 min were used as PI-positive control.

### Staining With Luminescent Conjugated Oligomers

The bacterial cultures were washed with PBS and the number of the bacteria were adjusted to 10⁸-10⁹ cells/mL. Bacteria were then treated with peptides (at sub-MIC or MIC concentration based on the aim of the study) or buffer for 2 h at 37° C. Then, cells were treated with LCO dyes (pFTAA; Amytacker™ 680 or Amytacker™ 545: final concentration of 0.5 μM; Ebba Biotech, Sweden) for at least 90 min. The absorption, emission and excitation spectra for each dye were measured based on the standard Ebbabiotec advice (ebbabiotech.com).

### Inclusion Body (IB) Purification

The overnight culture of bacteria was centrifuged for 30 min at 4,000×g and cells were washed with physiological water (NaCl 0.9%). Bacterial cells were treated by peptide at the appropriate concentration for at least 2 h at 37 C. The bacterial pellets were washed with 10 mL buffer A (50 mM HEPES, pH 7.5, 300 mM NaCl, 5 mM β-mercaptoethanol, 1.0 mM EDTA) and centrifuged at 4° C. for 30 min at 4,000×g. The supernatant was discarded and 20 mL of buffer B (buffer A plus 1 tablet of the protease and phosphatase Inhibitor Cocktail (ab201119, Abcam, UK) was added to the bacterial pellet. In order to break the cells, a High-Pressure Homogenizer (Glen Creston Ltd) with the pressure set to 20,000-25,000 psi was used on ice, and in addition, the suspensions were sonicated (Branson Digital sonifier 50/60 Hz) on ice with alternating 2 min cycle (15 pulses at 50% power with 30 s pauses on ice, until completing 2 min total sonication time). The lysed cells were centrifuged at 4° C. for 30 min at 11,000×g. The precipitated fraction was afterward re-suspended with 10 mL buffer D (buffer A plus 0.8% (V/V) Triton X-100, 0.1% sodium deoxycholate) and the suspension was sonicated to ensure the pellet is completely dissolved. This step was repeated three times. Centrifugation was performed at 4° C. for 30 min at 11,000×g. Finally, to solubilize IBs, the pellet was suspended in 500 ul of buffer F (50 mM HEPES, pH 7.5, 8.0 M urea) of precipitated fraction.

### SHV and TEM Protein Purification

Plasmids were prepared by Genscript (USA) vector construction services. DNA TEM (870 bp) or SHV (894 bp) were each sub-cloned into a PUC57 vector cloning site NdeI/XhoI, with an N-terminal HIS-tag followed by the TEV cleavage site. The proteins were expressed in E. coli BL21 (DE3) by inducing with 1 mM IPTG overnight at 20° C. Cells were harvested by centrifugation (15 minutes at 5000 rpm (2800×g) at 4° C. ), resuspended in buffer (500 mM Sucrose, 200 mM Tris pH8.5 plus protease inhibitors (mini ETDA free (SigmaAldrich), one tablet per 25 mL of buffer) and lysed using a high-pressure homogenizer (EmulsiFlex C5, Avestin, Canada). The cell debris was removed by centrifugation (30 minutes, 18×g) and the soluble lysate was loaded on a onto size exclusion chromatography (SEC) column 26/600 75 pg column (column vol 320 mL, GE Healthcare, USA). The protein was equilibrated with buffer 50 mM Tris pH 8.5, 300 mM NaCl.

### GFP Fusion Protein Construction

TEM- and SHV-GFP fusion proteins were subcloned into the Invitrogen pBAD myc/his A vector. To this end, a vector expressing GFP with a linker (sequence KPAGAAKGG) at its C-term designed in a previous study⁶⁴ was modified. A multiple cloning site containing EcoRI and SpeI restriction sites was introduced C-terminally of the linker sequence through site-directed mutagenesis (using the New England Biolabs Q5® Site-Directed Mutagenesis Kit). Next, SHV and TEM sequences with EcoRI and SpeI restriction sites at their N- and C-terminus, respectively, were produced through PCR amplification from the expression constructs used for purification (discussed above). Finally, both the vector and PCR inserts were digested with SpeI-HF® and EcoRI-HF® (New England Biolabs) and ligated according to the manufacturer's instructions.

Expression of TEM or SHV Fusion GFP in E. coli

For protein expression and solubility analysis, bacterial strains were grown overnight in Lysogeny Broth (LB Difco™) supplemented with ampicillin for GFP expression and both ampicillin and chloramphenicol for co-expression of the GFP constructs with pKJE7. The overnight cultures were diluted 1:100 in fresh LB supplemented with the appropriate antibiotics and grown to an OD of about 0.6, after which expression was induced with 0.2% arabinose. Expression was allowed to proceed for 3 hours after which cells were lysed in in B-PER™ reagent (ThermoFisher, USA) supplemented with 0.1 mg/ml lysozyme (Sigma-Aldrich), Complete™ Protease Inhibitor Cocktail (Sigma-Aldrich) and Pierce™ universal nuclease for cell lysis (ThermoFisher). Cells were lysed on ice for 30 mins, after which soluble and insoluble fractions were separated through centrifugation at 17.100×g for 30 mins at 4° C. Supernatant was removed and the insoluble fraction dissolved in an equal volume of 8M urea. GFP in soluble and insoluble fractions was then quantified through SDS-PAGE followed by Western blotting. Blots were developed using chemiluminescence after incubation with primary anti-GFP antibody (Antibody 2555S, Cell Signaling Technologies) or anti-DnaK antibody (D8076, USBio USA) and secondary HRP-conjugated antibody. Blots were quantified using Bio-Rad's Image Lab™ Software. Soluble GFP fractions were determined by calculating the ratio of soluble over total (soluble+insoluble) protein.

### Experimental Animals

Female C57BL/6Jax mice of 6 to 8 weeks with uniform weight (20 and 23 g) were used in this study (Harlan, The Netherlands). Mice were housed in plastic cages, four mice per cage on softwood granules as bedding. The room was kept between 21° C. and 25° C. with 12/12 h light-dark cycles. The animals had free access to water and pelleted rodent food. In order to avoid stress-induced confounding factors mice were transferred to the lab one week before experimental manipulation.

### Efficacy of TEM32 in the Treatment of Urinary Tract Infection in Mouse

To test the efficacy of the peptides, urinary tract infection model was performed as described previously⁶⁵. Briefly, female C57BL/6Jax mice female mice were deprived from water for at least one hour. Then, they were anesthetized by IP administration of the mixture of ketamine (Nimatek)/xylazine (XYL-M 2% BE-V170581). The bladder of the mouse was massaged with fingers and pushed down gently on to expel remaining urine. Mice were slowly inoculated urethrally with 50 μL of a bacterial suspension slowly (10⁸ CFU/mouse) using a sterile catheter (The plastic intravenous cannula of the paediatric intravenous-access cannula (GS391350) in the bladder over 5 s in order to avoid vesicoureteral reflux. The catheter was then removed directly after inoculation. After surgery, the animals were visually monitored for full recovery. After 1 h post-inoculation, all mice received ampicillin (30 mg/kg_PO-orally) and at the same time 3 groups of animals received the peptides via different administration routes (10mg/kg_IV—Intravenous; IP—Intraperitoneal or SC—sub-cutaneous) and the positive control groups received tazobactam (10 mg/Kg, PO). The negative control groups received vehicle or saline (IV administration). 2 h post inoculation, all mice received a second injection with the same concentration of each treatment as explained above. Twenty-four hours post infection, mice were sacrificed and organs (kidney, bladder, ureter) washed with PBS and were homogenized (Thermo Savant FastPrep FP120 Homogenizer/24 s). The homogenized tissues were serially diluted and cultured on blood agar plates. The plates were incubated overnight at 37° C. and the number of bacteria was measured by CFU value.

### Structured Illumination Microscopy (SIM)

Bacteria were fixed by adding 2.5% paraformaldehyde and 0.04% glutaraldehyde (final concentrations) to the culture media, followed by incubation at room temperature for 15 mins and 30 mins on ice. Bacteria were then washed in PBS and resuspended in GTE buffer (50 mM glucose, 25 mM Tris, and 10 mM EDTA, pH 8.0). Directly preceding microscopic analysis, cells were transferred to a glass slide and covered with a coverslip. Imaging was performed using a Zeiss Elyra S.1 system in the LiMoNe Light microscopy facility of VIB-KU Leuven.

### Statistics

Statistical analysis was performed using Prism or R. Unpaired student's t test, one-sample t test and ANOVA were used to determine significant differences between samples unless otherwise indicated. Significance levels: * for P<0,05; ** for P<0,01; *** for P<0,001; **** for P<0,0001. Non-significant differences are not separately labelled, unless stated otherwise.

### Tables

TABLE 1
APRs identified by TANGO in E. coli TEM
β-lactamase (UniProt Accession BLAT_ECOLX)
and the resulting peptides tested.

| | posi- | | TANGO | | adjusted | Peptide |
|---|---|---|---|---|---|---|
| # | tion | APR | score | length | APR | sequence |
| 1 | 12 | FFAAFC | 46.5 | 6 | FFAAFC | RFFAAFCL |
| | | (SEQ ID | | | L | RRRPFFAA |
| | | NO: 15) | | | (SEQ ID | FCLRR |
| | | | | | NO: 16) | (SEQ ID |
| | | | | | | NO: 27) |
| 2 | 134 | LLLTTI | 43.6 | 6 | LLLTTI | RLLLTTIG |
| | | (SEQ ID | | | G | RRPRLLLT |
| | | NO: 17) | | | (SEQ ID | TIGRR |
| | | | | | NO: 18) | (SEQ ID |
| | | | | | | NO: 28) |
| 3 | 145 | LTAFL | 31.9 | 5 | LTAFLHN | RLTAFLHN |
| | | (SEQ ID | | | (SEQ ID | RRPRLTAF |
| | | NO: 11) | | | NO: 9) | LHNRR |
| | | | | | | (SEQ |
| | | | | | | ID NO: 5) |
| 4 | 195 | LLTLA | 18.9 | 5 | LLTLAS | RLLTLASR |
| | | (SEQ ID | | | (SEQ ID | RPRLLTLA |
| | | NO: 19) | | | NO: 20) | SRR |
| | | | | | | (SEQ ID |
| | | | | | | NO: 29) |
| 5 | 224 | AGWFIA | 14.5 | 6 | AGWFIA | RAGWFIAR |
| | | | | | (SEQ ID | RPRAGWFI |
| | | (SEQ ID | | | NO: 22) | ARR |
| | | NO: 21) | | | | (SEQ ID |
| | | | | | | NO: 30) |
| 6 | 242 | IIAAL | 11.1 | 5 | GIIAALG | RGIIAALG |
| | | (SEQ ID | | | (SEQ ID | RRPRGIIA |
| | | NO: 23) | | | NO: 24) | ALGR |
| | | | | | | R (SEQ ID |
| | | | | | | NO: 31) |
| 7 | 255 | IVVIYTT | 78.9 | 7 | IVVIYTT | RIVVIYT |
| | | | | | (SEQ ID | TRRPRIVVI |
| | | (SEQ ID | | | NO: 26) | YTTR |
| | | NO: 25) | | | | R (SEQ ID |
| | | | | | | NO: 32) |

TABLE 2
MIC values of the TEM peptides as well as penicillin in the presence
peptide or tazobactam for E. coli strain UZ_TEM104.

| | | concentration | |
|---|---|---|---|
| | MIC peptide | of additive | MIC penicillin |
| peptide | (μg/mL) | (μg/mL) | (μg/mL) |
| None (buffer) | — | 0 | 1600 |
| TEM1.0 | >100 | 50 | 1600 |
| TEM2.0 | >100 | 50 | 800 |
| TEM3.0 | >100 | 50 | 200 |
| TEM4.0 | 100 | 50 | 1600 |
| TEM5.0 | 100 | 50 | 1600 |
| TEM6.0 | >100 | 50 | 1600 |
| TEM7.0 | >100 | 50 | 1600 |
| tazobactam | — | 50 | 400 |
| Tazobactam + TEM3.0 | — | 25 + 25 | 50 |

TABLE 3
MIC values of penicillin for various clinical isolates of E. coli
in the absence or presence of 30 μg/mL of peptide TEM3.2

| | | | | | | MIC |
|---|---|---|---|---|---|---|
| | | | | | | ampicillin + |
| year | Country | | | β- | MIC | 30 μg/mL |
| collected | of origin | Infection | Organ | lactamase | ampicillin | TEM-3.2 |
| NA | NA | NA | | TEM-1 | >64 | ≤0.06 |
| NA | NA | NA | | TEM-1 | >64 | ≤0.06 |
| NA | NA | NA | | TEM-1 | >64 | ≤0.06 |
| 2016 | South | IAI | Peritoneal | TEM-ESBL | >64 | ≤0.06 |
| | Africa | | Fluid | | | |
| 2016 | South | IAI | Peritoneal | TEM-ESBL | >64 | ≤0.06 |
| | Africa | | Fluid | | | |
| 2016 | Germany | UTI | Urine | TEM-ESBL | >64 | ≤0.06 |
| 2017 | Israel | UTI | Urine | TEM-ESBL | >64 | ≤0.06 |
| 2017 | Portugal | RTI | Bronchi | TEM-ESBL | >64 | 0.5 |
| 2016 | Lithuania | UTI | Urine | TEM-ESBL | >64 | 1 |
| 2017 | Germany | RTI | Bronchi | SHV-12; TEM-ESBL; | >64 | 0.12 |
| 2017 | Turkey | UTI | Urine | SHV-2; CTX-M-55; | >64 | ≤0.06 |
| | | | | VIM-1; | | |
| 2017 | Turkey | IAI | Abscess | TEM-ESBL; CTX-M-24; | >64 | ≤0.06 |
| | | | | OXA-48 | | |
| 2017 | France | IAI | Peritoneal | TEM-ESBL; CTX-M- | >64 | ≤0.06 |
| | | | Fluid | TYPE; | | |
| 2017 | Romania | UTI | Urine | TEM-ESBL; CTX-M-27; | >64 | ≤0.06 |
| 2017 | Qatar | UTI | Urine | TEM-ESBL; CTX-M-15; | >64 | >64 |
| | | | | NDM-5; | | |
| 2017 | Qatar | IAI | Abscess | TEM-ESBL; CTX-M-15; | >64 | >64 |
| | | | | NDM-19; | | |

UTI—Urinary Tract Infection,
IAI—Intra-abdominal Infection,
RTI—Respiratory Tract Infection

TABLE 4
The antibiotic resistance profiles of bacterial strains used in the present
invention. VITEK ® 2 AST Cards used for antimicrobial susceptibility testing.

| | E. coli | | E. coli | | E. coli | | E. coli | |
|---|---|---|---|---|---|---|---|---|
| Antimicrobial | TEM-104 | | TEM-206 | | TEM-30 | | TEM-148 | |
| agent | MIC | Inter [symbol] tion | MIC | Inter [symbol] tion | MIC | Inter [symbol] tion | MIC | Inter [symbol] tion |
| Ampicillin | >=32 | R | >=32 | R | >=32 | R | >=32 | R |
| Amoxicillin + | >=32 | R | 4 | [symbol] R | 16 | [symbol] R | 4 | [symbol] R |
| cla [symbol] anic acid | | | | | | | | |
| Piperacillin/ | 16 | [symbol] | <=4 | [symbol] R | <=4 | [symbol] R | <=4 | [symbol] R |
| ta [symbol] bactam | | | | | | | | |
| T [symbol] cillin | 8 | S | [symbol] | S | <=4 | S | <=4 | S |
| C [symbol] xime | 4 | S | >=64 | R | >=64 | R | >=64 | R |
| C [symbol] | 4 | S | >=64 | R | >=64 | R | >=64 | R |
| A [symbol] | | | | | | | | |
| Cef [symbol] axime | <=1 | S | >=64 | R | 4 | R | 16 | R |
| C [symbol] dime | <=1 | S | 16 | R | <=1 | [symbol] I | <=1 | [symbol] I |
| C [symbol] ime | <=1 | S | >=64 | R | <=1 | [symbol] I | 2 | [symbol] I |
| C [symbol] tin | <=4 | S | <=4 | S | 8 | S | <=4 | S |
| Meropen [symbol] m | [symbol] | S | <=0.25 | S | <=0.25 | S | <=0.25 | S |
| Levofloxacin | <0.12 | S | [symbol] | R | [symbol] | R | [symbol] | S |
| Nitrofuran [symbol] | [symbol] | S | <=18 | S | 32 | S | 32 | S |
| Gentamicin | <=1 | S | <=1 | S | >=16 | R | <=1 | S |
| Tobramycin | <=1 | S | <=1 | S | 8 | [symbol] R | <=1 | S |
| Amikacin | <=2 | S | <=2 | S | <=2 | S | <=2 | S |
| Trimethoprim/ | <=20 | S | >=320 | R | 40 | S | >=320 | R |
| sulfamethoxaz [symbol] | | | | | | | | |
| Tig [symbol] cycline | <=0.5 | S | <=0.5 | S | 2 | I | <=0.5 | S |
| Colistin | <=0.5 | S | <=0.5 | S | <=0.5 | S | <=0.5 | S |
| | E. coli | | E. coli | | E. coli | | E. coli | |
| Antimicrobial | TEM-1 [symbol] | | NDM [symbol] a | | NDM [symbol] b | | SH [symbol] (APHA) | |
| agent | MIC | Inter [symbol] tion | MIC | Inter [symbol] tion | MIC | Inter [symbol] tion | MIC | Inter [symbol] tion |
| Ampicillin | >=32 | R | >=32 | R | >=32 | R | >=32 | R |
| Amoxicillin + | 16 | [symbol] R | >=32 | R | >=32 | R | [symbol] | R |
| cla [symbol] anic acid | | | | | | | | |
| Piperacillin/ | <=4 | [symbol] R | >=128 | R | >=128 | R | >=128 | R |
| ta [symbol] bactam | | | | | | | | |
| T [symbol] cillin | >=32 | R | >=32 | R | >=32 | R | <=4 | R |
| C [symbol] xime | >=64 | R | >=64 | R | >=64 | R | [symbol] | R |
| C [symbol] | >=64 | R | >=64 | R | >=64 | R | [symbol] | R |
| A [symbol] | | | | | | | | |
| Cef [symbol] axime | >=64 | R | >=64 | R | >=64 | R | >=64 | R |
| C [symbol] dime | 16 | R | >=64 | R | >=64 | R | >=64 | R |
| C [symbol] ime | >=64 | R | >=64 | R | >=64 | R | 16 | R |
| C [symbol] tin | 8 | S | >32 | R | >32 | R | >32 | R |
| Meropen [symbol] m | <=0.25 | S | >=16 | R | 8 | I | <=0.25 | S |
| Levofloxacin | <=0.12 | S | >=8 | R | >=8 | R | <=0.12 | S |
| Nitrofuran [symbol] | <=16 | S | 64 | R | 64 | R | <=16 | S |
| Gentamicin | <=1 | S | >=16 | R | <=1 | S | [symbol] | R |
| Tobramycin | <=1 | S | 8 | R | <=1 | S | 4 | [symbol] R |
| Amikacin | <=2 | S | <=2 | S | <=2 | S | <=2 | S |
| Trimethoprim/ | <=20 | S | <=20 | S | >=320 | R | >=320 | R |
| sulfamethoxaz [symbol] | | | | | | | | |
| Tig [symbol] cycline | <0.5 | S | <0.5 | S | <=0.5 | S | <=0.5 | S |
| Colistin | <=0.5 | S | <=0.5 | S | <=0.5 | S | <=0.5 | S |

Abbreviations: S, susceptible; I, intermediate; R, resistant. [symbol]
indicates data missing or illegible when filed

### REFERENCES

1 Abraham, E. P. & Chain, E. An enzyme from bacteria able to destroy penicillin. Nature 146, 837 (1940).
2 Bradford, P. A. Extended-spectrum beta-lactamases in the 21st century: characterization, epidemiology, and detection of this important resistance threat. Clin Microbiol Rev 14, 933-951, table of contents, doi:10.1128/CMR.14.4.933-951.2001 (2001).
3 Van Puyvelde, S., Deborggraeve, S. & Jacobs, J. Why the antibiotic resistance crisis requires a One Health approach. Lancet Infect Dis, doi:10.1016/S1473-3099(17)30704-1 (2017).
4 Naas, T. et al. Beta-lactamase database (BLDB)—structure and function. J Enzyme Inhib Med Chem 32, 917-919, doi:10.1080/14756366.2017.1344235 (2017).
5 Bush, K. & Jacoby, G. A. Updated functional classification of beta-lactamases. Antimicrobial agents and chemotherapy 54, 969-976, doi: 10.1128/AAC.01009-09 (2010).
6 Drawz, S. M. & Bonomo, R. A. Three decades of beta-lactamase inhibitors. Clin Microbiol Rev 23, 160-201, doi:10.1128/CMR.00037-09 (2010).
7 Datta, N. & Kontomichalou, P. Penicillinase synthesis controlled by infectious R factors in Enterobacteriaceae. Nature 208, 239-241 (1965).
8 Salverda, M. L., De Visser, J. A. & Barlow, M. Natural evolution of TEM-1 beta-lactamase: experimental reconstruction and clinical relevance. FEMS Microbiol Rev 34, 1015-1036, doi:10.1111/j.1574-6976.2010.00222.x (2010).
9 Paterson, D. L. & Bonomo, R. A. Extended-spectrum beta-lactamases: a clinical update. Clin Microbiol Rev 18, 657-686, doi:10.1128/CMR.18.4.657-686.2005 (2005).
10 Langenberg, T., Schymkowitz, J. & Rousseau, F. Identifying rescuers of misfolding. Nat Biomed Eng 1, 782-783, doi: 10.1038/s41551-017-0149-y (2017).
11 Liguori, L. et al. Pharmacological Chaperones: A Therapeutic Approach for Diseases Caused by Destabilizing Missense Mutations. Int J Mol Sci 21, doi:10.3390/ijms21020489 (2020).
12 Siekierska, A. et al. alpha-Galactosidase aggregation is a determinant of pharmacological chaperone efficacy on Fabry disease mutants. J Biol Chem 287, 28386-28397, doi:10.1074/jbc.M112.351056 (2012).
13 Rozenfeld, P. & Neumann, P. M. Treatment of fabry disease: current and emerging strategies. Curr Pharm Biotechnol 12, 916-922, doi:1389-2010/11 $58.00+.00 (2011).
14 Bulawa, C. E. et al. Tafamidis, a potent and selective transthyretin kinetic stabilizer that inhibits the amyloid cascade. Proc Natl Acad Sci U S A 109, 9629-9634, doi:10.1073/pnas.1121005109 (2012).
15 Eisenberg, D. & Jucker, M. The amyloid state of proteins in human diseases. Cell 148, 1188-1203, doi:10.1016/j.cell.2012.02.022 (2012).
16 Ganesan, A. et al. Selectivity of aggregation-determining interactions. J Mol Biol 427, 236-247, doi:10.1016/j.jmb.2014.09.027 (2015).
17 Rousseau, F., Serrano, L. & Schymkowitz, J. W. How evolutionary pressure against protein aggregation shaped chaperone specificity. J Mol Biol 355, 1037-1047, doi:10.1016/j.jmb.2005.11.035 (2006).
18 Linding, R., Schymkowitz, J., Rousseau, F., Diella, F. & Serrano, L. A comparative study of the relationship between protein structure and beta-aggregation in globular and intrinsically disordered proteins. J Mol Biol 342, 345-353, doi:10.1016/j.jmb.2004.06.088 (2004).
19 Bednarska, N. G. et al. Protein aggregation as an antibiotic design strategy. Mol Microbiol, doi:10.1111/mmi.13269 (2015).
20 Khodaparast, L. et al. Aggregating sequences that occur in many proteins constitute weak spots of bacterial proteostasis. Nature communications 9, 866, doi:10.1038/s41467-018-03131-0 (2018).
21 Betti, C. et al. Sequence-Specific Protein Aggregation Generates Defined Protein Knockdowns in Plants. Plant physiology 171, 773-787, doi:10.1104/pp.16.00335 (2016).
22 Betti, C., Schymkowitz, J., Rousseau, F. & Russinova, E. Selective Knockdowns in Maize by Sequence-Specific Protein Aggregation. Methods Mol Biol 1676, 109-127, doi:10.1007/978-1-4939-7315-6_6 (2018).
23 Gallardo, R. et al. De novo design of a biologically active amyloid. Science 354, doi:10.1126/science.aah4949 (2016).
24 Torrent, M., Valle, J., Nogues, M. V., Boix, E. & Andreu, D. The Generation of Antimicrobial Peptide Activity: A Trade-off between Charge and Aggregation? Angewandte Chemie 123, 10874-10877, doi:10.1002/ange.201103589 (2011).
25 Last, N. B. & Miranker, A. D. Common mechanism unites membrane poration by amyloid and antimicrobial peptides. Proc Natl Acad Sci U S A 110, 6382-6387, doi:10.1073/pnas.1219059110 (2013).
26 Torrent, M., Andreu, D., Nogues, V. M. & Boix, E. Connecting Peptide Physicochemical and Antimicrobial by Prediction Model. Properties by a Rational Prediction Model. PLOS One 6, doi:10.1371/journal.pone.0016968 (2011).
27 Torrent, M., Odorizzi, F., Nogues, M. V. & Boix, E. Eosinophil cationic protein aggregation: identification of an N-terminus amyloid prone region. Biomacromolecules 11, 1983-1990, doi:10.1021/bm100334u (2010).
28 Jang, H. et al. Antimicrobial protegrin-1 forms amyloid-like fibrils with rapid kinetics suggesting a functional link. Biophys J 100, 1775-1783, doi:10.1016/j.bpj.2011.01.072 (2011).
29 Soscia, S. J. et al. The Alzheimer's Disease-Associated Amyloid beta-Protein Is an Antimicrobial Peptide. Plos One 5, doi:ARTN e950510.1371/journal.pone.0009505 (2010).
30 Harris, F., Dennison, S. R. & Phoenix, D. A. Aberrant action of amyloidogenic host defense peptides: a new paradigm to investigate neurodegenerative disorders? FASEB J 26, 1776-1781, doi:10.1096/fj.11-199208 (2012).
31 Gosztyla, M. L., Brothers, H. M. & Robinson, S. R. Alzheimer's Amyloid-beta is an Antimicrobial Peptide: A Review of the Evidence. Journal of Alzheimer's disease: JAD 62, 1495-1506, doi:10.3233/JAD-171133 (2018).
32 Kumar, D. K. et al. Amyloid-beta peptide protects against microbial infection in mouse and worm models of Alzheimer's disease. Science translational medicine 8, 340ra372, doi:10.1126/scitranslmed.aaf1059 (2016).
33 Fernandez-Escamilla, A. M., Rousseau, F., Schymkowitz, J. & Serrano, L. Prediction of sequence-dependent and mutational effects on the aggregation of peptides and proteins. Nature Biotechnology 22, 1302-1306, doi:10.1038/nbt1012 (2004).
34 Chandonia, J. M., Fox, N. K. & Brenner, S. E. SCOPe: Manual Curation and Artifact Removal in the Structural Classification of Proteins—extended Database. J Mol Biol 429, 348-355, doi:10.1016/j.jmb.2016.11.023 (2017).
35 Fu, L., Niu, B., Zhu, Z., Wu, S. & Li, W. CD-HIT: accelerated for clustering the next-generation sequencing data. Bioinformatics 28, 3150-3152, doi:10.1093/bioinformatics/bts565 (2012).
36 Naas, T. et al. Beta-lactamase database (BLDB)—structure and function. Journal of Enzyme Inhibition and Medicinal Chemistry 32, 917-919, doi:10.1080/14756366.2017.1344235 (2017).
37 Salverda, M. L. M., De Visser, J. A. G. M. & Barlow, M. Natural evolution of TEM-1 β-lactamase: experimental reconstruction and clinical relevance. FEMS Microbiology Reviews 34, 1015-1036, doi:10.1111/j.1574-6976.2010.00222.x (2010).
38 Brown, N. G., Pennington, J. M., Huang, W., Ayvaz, T. & Palzkill, T. Multiple Global Suppressors of Protein Stability Defects Facilitate the Evolution of Extended-Spectrum TEM β-Lactamases. 404, 832-846, doi:10.1016/j.jmb.2010.10.008 (2010).
39 Abriata, L. A., M. Salverda, M. L. & Tomatis, P. E. Sequence-function-stability relationships in proteins from datasets of functionally annotated variants: The case of TEM β-lactamases. 586, 3330-3335, doi:10.1016/j.febslet.2012.07.010 (2012).
40 Cantu, C., 3rd & Palzkill, T. The role of residue 238 of TEM-1 beta-lactamase in the hydrolysis of extended-spectrum antibiotics. J Biol Chem 273, 26603-26609, doi:10.1074/jbc.273.41.26603 (1998).
41 Docquier, J. D. et al. Crystal structure of the OXA-48 beta-lactamase reveals mechanistic diversity among class D carbapenemases. Chemistry & biology 540-547, 16, doi:10.1016/j.chembiol.2009.04.010 (2009).
42 Franceschini, N. et al. Purification and biochemical characterization of the VIM-1 metallo-beta-lactamase. Antimicrobial agents and chemotherapy 44, 3003-3007, doi:10.1128/aac.44.11.3003-3007.2000 (2000).
43 Kumar, G., Issa, B., Kar, D., Biswal, S. & Ghosh, A. S. E152A substitution drastically affects NDM-5 activity. FEMS Microbiol Lett 364, doi:10.1093/femsle/fnx008 (2017).
44 Cremers, C. M. et al. Polyphosphate: A Conserved Modifier of Amyloidogenic Processes. Mol Cell 63, 768-780, doi:10.1016/j.molcel.2016.07.016 (2016).
45 Klingstedt, T. et al. Synthesis of a library of oligothiophenes and their utilization as fluorescent ligands for spectral assignment of protein aggregates. Organic & biomolecular chemistry 9, 8356-8370, doi:10.1039/c1ob05637a (2011).
46 Hammarstrom, P. et al. A fluorescent pentameric thiophene derivative detects in vitro-formed prefibrillar protein aggregates. Biochemistry 49, 6838-6845, doi:10.1021/bi100922r (2010).
47 Aslund, A. et al. Novel pentameric thiophene derivatives for in vitro and in vivo optical imaging of a plethora of protein aggregates in cerebral amyloidoses. ACS chemical biology 4, 673-684, doi:10.1021/cb900112v (2009).
48 Khodaparast, L. et al. Aggregating sequences that occur in many proteins constitute weak spots of bacterial proteostasis. Nature communications 9, 866, doi: 10.1038/s41467-018-03131-0 (2018).
49 Wang, X., Lu, M., Shi, Y., Ou, Y. & Cheng, X. Discovery of novel new Delhi metallo-beta-lactamases-1 inhibitors by multistep virtual screening. PLOS One 10, e0118290, doi:10.1371/journal.pone.0118290 (2015).
50 Yong, D. et al. Characterization of a new metallo-beta-lactamase gene, bla(NDM-1), and a novel erythromycin esterase gene carried on a unique genetic structure in Klebsiella pneumoniae sequence type 14 from India. Antimicrobial agents and chemotherapy 53, 5046-5054, doi:10.1128/AAC.00774-09 (2009).
51 Kumarasamy, K. K. et al. Emergence of a new antibiotic resistance mechanism in India, Pakistan, and the UK: a molecular, biological, and epidemiological study. Lancet Infect Dis 10, 597-602, doi:10.1016/S1473-3099(10)70143-2 (2010).
52 Khan, A. U., Maryam, L. & Zarrilli, R. Structure, Genetics and Worldwide Spread of New Delhi Metallo-beta-lactamase (NDM): a threat to public health. BMC Microbiol 17, 101, doi:10.1186/s12866-017-1012-8 (2017).
53 Maurer-Stroh, S. et al. Exploring the sequence determinants of amyloid structure using position-specific scoring matrices. Nature Methods 7, 237-U109, doi:10.1038/Nmeth.1432 (2010). Hede, K. Antibiotic resistance: An infectious arms race. Nature 509, S2-3, doi:10.1038/509S2a 54 (2014).
55 UniProt, C. The universal protein resource (UniProt). Nucleic Acids Res 36, D190-195, doi:10.1093/nar/gkm895 (2008).
56 Abriata, L. A., Salverda, M. L. & Tomatis, P. E. Sequence-function-stability relationships in proteins from datasets of functionally annotated variants: the case of TEM beta-lactamases. FEBS Lett 586, 3330-3335, doi:10.1016/j.febslet.2012.07.010 (2012).
57 Brown, N. G., Pennington, J. M., Huang, W., Ayvaz, T. & Palzkill, T. Multiple global suppressors of protein stability defects facilitate the evolution of extended-spectrum TEM beta-lactamases. J Mol Biol 404, 832-846, doi:10.1016/j.jmb.2010.10.008 (2010).
58 Schymkowitz, J. et al. The FoldX web server: an online force field. Nucleic Acids Res 33, W382-388, doi:10.1093/nar/gki387 (2005).
59 Fonze, E. et al. TEM1 beta-lactamase structure solved by molecular replacement and refined structure of the S235A mutant. Acta crystallographica. Section D, Biological crystallography 51, 682-694, doi:10.1107/S0907444994014496 (1995).
60 Krieger, E. & Vriend, G. YASARA View—molecular graphics for all devices—from smartphones to workstations. Bioinformatics 30, 2981-2982, doi:10.1093/bioinformatics/btu426 (2014).
61 Sirot, J. Detection of extended-spectrum plasmid-mediated beta-lactamases by disk diffusion. Clin Microbiol Infect 2 Suppl 1, S35-S39, doi: 10.1111/j.1469-0691.1996.tb00873.x (1996).
62 Hall, M. J., Middleton, R. F. & Westmacott, D. The fractional inhibitory concentration (FIC) index as a measure of synergy. Journal of Antimicrobial Chemotherapy 11, 427-433 (1983).
63 Botelho, M. G. Fractional inhibitory concentration index of combinations of antibacterial agents against cariogenic organisms. J Dent 28, 565-570, doi:10.1016/s0300-5712(00)00039-7 (2000).
64 Beerten, J. et al. Aggregation gatekeepers modulate protein homeostasis of aggregating sequences and affect bacterial fitness. Protein Engineering Design & Selection 25, 357-366, doi:10.1093/protein/gzs031 (2012).
65 Thai, K. H., Thathireddy, A. & Hsieh, M. H. Transurethral induction of mouse urinary tract infection. J Vis Exp, doi:10.3791/2070 (2010).

1. A non-naturally occurring molecule comprising:
a. NGK1-P1-CGK1,
b. NGK1-P1-CGK1-Z1-NGK2-P2-CGK2, or
c. NGK1-P1-CGK1-Z1-NGK2-P2-CGK2-Z2-NGK3-P3-CGK3,
wherein:
P1 to P3 each independently denote an amino acid stretch comprising
i)
(SEQ ID NO: 1)
LTAFLX₁X₂,
wherein X₁ is H or R
and
X₂ is N or Q
or
ii)
(SEQ ID NO: 2)
TAQILNW,
or
iii)
(SEQ ID NO: 3)
AQILNWI,
or
iv)
(SEQ ID NO: 4)
LAAALML;
NGK1, NGK2, NGK3; CGK1, CGK2, and CGK3 each independently denote 1 to 3 contiguous amino acids that display low beta-sheet potential or a propensity to disrupt beta-sheets, and Z1 and Z2 each independently denote a linker.

2. The molecule of claim 1, wherein each linker is independently selected from a stretch between 1 and 5 units, wherein each unit is independently an amino acid or PEG.

3. The molecule of claim 1, wherein the molecule comprises a peptide of the amino acid sequence:
a.
(SEQ ID NO: 5)
RLTAFLHNRRPRLTAFLHNRR,
or
b.
(SEQ ID NO: 6)
RLTAFLRQRRPRLTAFLRQRR,
or
c.
(SEQ ID NO: 7)
RLTAFLHNRRPRLTAFLRQRR,
or
d.
(SEQ ID NO: 8)
RLTAFLRQRRPRLTAFLHNRR,

4. A non-naturally occurring molecule configured to form an intermolecular beta-sheet with an extended-spectrum beta-lactamase of class A wherein the intermolecular beta-sheet involves:
a. the amino acid sequence LTAFLHN (SEQ ID NO: 9) present in the extended-spectrum beta-lactamase protein of class A and/or
b. the amino acid sequence LTAFLRQ (SEQ ID NO: 10) present in the extended-spectrum beta-lactamase protein of class A.

5. The molecule of claim 4 wherein the amino acid sequence comprises one or more D-amino acids and/or amin acid analogue.

6. The molecule of claim 1, wherein the molecule comprises a detectable label, a moiety that allows for isolation of the molecule, a moiety increasing the stability or half-life of the molecule, a moiety increasing the solubility of the molecule, and/or a moiety increasing the bacterial uptake of the molecule.

7. The molecule of claim 1 in combination with a beta-lactam antibiotic.

8. (canceled)

9. A method of treating a bacterial infection, the method comprising administering to the infection the molecule of claim 1.

10. The molecule of claim 1, wherein the molecule is comprised in a pharmaceutical composition.

11. The molecule of claim 7, wherein the combination is comprised in a pharmaceutical composition or a kit of parts.

12. The molecule of claim 1, wherein the molecule is configured to form an intermolecular beta-sheet with an extended-spectrum beta-lactamase of class A.

13. The molecule of claim 1, wherein the 1 to 3 contiguous amino acids are selected from the group consisting of R, K, E, D, and P, D-isomers and/or analogues thereof, and combinations thereof.

14. The molecule of claim 2, wherein each linker is independently GS, P, PP, or D-isomers and/or analogues thereof.

15. The molecule of claim 3, wherein the amino acid sequence comprises one or more D-amino acids and/or analogues of one or more of its amino acids.

16. The molecule of claim 3, wherein the N-terminal amino acid is acetylated and/or the C-terminal amino acid is amidated.

17. The molecule of claim 7, wherein the beta-lactam antibiotic is selected from the group consisting of penicillin derivatives, cephems, penems, monobactams, clavams, carbacephems, and oxacephems.
